SEQUENCE LISTING (68 sequences)

SEQ ID NO: 1 - DNA, 360 bp, synthetic construct
  misc_feature 1..360: note = synthesized
  source 1..360: mol_type = other DNA
gaggtgcagctggtggagtctgggggaggcttggtccagcctggggggtccctgagactctcctgtgcagcctctggattcaccatcagtaccaatgcaatgagctgggtccgccaggctccagggaaggggctggagtggatcggagtcattactggtcgtgatatcacatactacgcgagctgggcgaaaggcagattcaccatctccagagacaattccaagaacacgctgtatcttcaaatgaacagcctgagagccgaggacacggctgtgtattactgtgcgcgcgacggtggatcatctgctattactagtaacaacatttggggccaaggaactctggtcaccgtttcttca

SEQ ID NO: 2 - protein, 120 aa, synthetic construct
  REGION 1..120: note = synthesized
EVQLVESGGGLVQPGGSLRLSCAASGFTISTNAMSWVRQAPGKGLEWIGVITGRDITYYASWAKGRFTISRDNSKNTLYLQMNSLRAEDTAVYYCARDGGSSAITSNNIWGQGTLVTVSS

SEQ ID NO: 3 - DNA, 336 bp, synthetic construct
  misc_feature 1..336: note = synthesized
  source 1..336: mol_type = other DNA
gacgtcgtgatgacccagtctccttccaccctgtctgcatctgtaggagacagagtcaccatcaattgccaagccagtgagagcattagcagttggttagcctggtatcagcagaaaccagggaaagcccctaagctcctgatctatgaagcatccaaactggcatctggggtcccatcaaggttcagcggcagtggatctgggacagagttcactctcaccatcagcagcctgcagcctgatgattttgcaacttattactgccaaggctatttttattttattagtcgtacttatgtaaattctttcggcggagggaccaaggtggagatcaaa

SEQ ID NO: 4 - protein, 112 aa, synthetic construct
  REGION 1..112: note = synthesized
DVVMTQSPSTLSASVGDRVTINCQASESISSWLAWYQQKPGKAPKLLIYEASKLASGVPSRFSGSGSGTEFTLTISSLQPDDFATYYCQGYFYFISRTYVNSFGGGTKVEIK

SEQ ID NO: 5 - DNA, 360 bp, synthetic construct
  misc_feature 1..360: note = synthesized
  source 1..360: mol_type = other DNA
gaggtgcagctggtggagtctgggggaggcttggtccagcctggggggtccctgagactctcctgtgcagcctctggaatcgacctcagtagcaatgcaatgagctgggtccgccaggctccagggaaggggctggagtggatcggagtcattactggtcgtgatatcacatactacgcgagctgggcgaaaggcagattcaccatctccagagacaattccaagaacacgctgtatcttcaaatgaacagcctgagagccgaggacacggctgtgtattactgtgcgcgcgacggtggatcatctgctattaatagtaagaacatttggggccaaggaactctggtcaccgtttcttca

SEQ ID NO: 6 - protein, 120 aa, synthetic construct
  REGION 1..120: note = synthesized
EVQLVESGGGLVQPGGSLRLSCAASGIDLSSNAMSWVRQAPGKGLEWIGVITGRDITYYASWAKGRFTISRDNSKNTLYLQMNSLRAEDTAVYYCARDGGSSAINSKNIWGQGTLVTVSS

SEQ ID NO: 7 - DNA, 336 bp, synthetic construct
  misc_feature 1..336: note = synthesized
  source 1..336: mol_type = other DNA
gacatccagatgacccagtctccttccaccctgtctgcatctgtaggagacagagtcaccatcacttgccaagccagtgagagcattagcagttggttagcctggtatcagcagaaaccagggaaagcccctaagctcctgatctatgaagcatccaaactggcatctggggtcccatcaaggttcagcggcagtggatctgggacagagttcactctcaccatcagcagcctgcagcctgatgattttgcaacttattactgccaaggctatttttattttattagtcgtacttatgtaaatgctttcggcggagggaccaaggtggagatcaaa

SEQ ID NO: 8 - protein, 112 aa, synthetic construct
  REGION 1..112: note = synthesized
DIQMTQSPSTLSASVGDRVTITCQASESISSWLAWYQQKPGKAPKLLIYEASKLASGVPSRFSGSGSGTEFTLTISSLQPDDFATYYCQGYFYFISRTYVNAFGGGTKVEIK

SEQ ID NO: 9 - DNA, 345 bp, synthetic construct
  misc_feature 1..345: note = synthesized
  source 1..345: mol_type = other DNA
cagtcggtggaggagtctgggggaggcttggtccagcctggggggtccctgagactctcctgtacagcctctggaatcgaccttaatacctacgacatgatctgggtccgccaggctccaggcaaggggctagagtgggttggaatcattacttatagtggtagtagatactacgcgaactgggcgaaaggccgattcaccatctccaaagacaataccaagaacacggtgtatctgcaaatgaacagcctgagagctgaggacacggctgtgtattactgtgccagagattatatgagtggttcccacttgtggggccagggaaccctggtcaccgtctctagt

SEQ ID NO: 10 - protein, 115 aa, synthetic construct
  REGION 1..115: note = synthesized
QSVEESGGGLVQPGGSLRLSCTASGIDLNTYDMIWVRQAPGKGLEWVGIITYSGSRYYANWAKGRFTISKDNTKNTVYLQMNSLRAEDTAVYYCARDYMSGSHLWGQGTLVTVSS

SEQ ID NO: 11 - DNA, 330 bp, synthetic construct
  misc_feature 1..330: note = synthesized
  source 1..330: mol_type = other DNA
gcctatgatatgacccagtctccatcttccgtgtctgcatctgtaggagacagagtcaccatcaagtgtcaggccagtgaggacatttatagcttcttggcctggtatcagcagaaaccagggaaagcccctaagctcctgatccattctgcatcctctctggcatctggggtcccatcaaggttcagcggcagtggatctgggacagatttcactctcaccatcagcagcctgcagcctgaagattttgcaacttactattgtcaacagggttatggtaaaaataatgttgataatgctttcggcggagggaccaaggtggagatcaaa

SEQ ID NO: 12 - protein, 110 aa, synthetic construct
  REGION 1..110: note = synthesized
AYDMTQSPSSVSASVGDRVTIKCQASEDIYSFLAWYQQKPGKAPKLLIHSASSLASGVPSRFSGSGSGTDFTLTISSLQPEDFATYYCQQGYGKNNVDNAFGGGTKVEIK

SEQ ID NO: 13 - DNA, 366 bp, synthetic construct
  misc_feature 1..366: note = synthesized
  source 1..366: mol_type = other DNA
gaggtgcagctgttggagtctgggggaggcttggtacagcctggggggtccctgagactctcctgtgcagcctctggattctccttcagtagcgggtacgacatgtgctgggtccgccaggctccagggaaggggctggagtggatcgcatgcattgctgctggtagtgctggtatcacttacgacgcgaactgggcgaaaggccggttcaccatctccagagacaattccaagaacacgctgtatctgcaaatgaacagcctgagagccgaggacacggccgtatattactgtgcgagatcggcgttttcgttcgactacgccatggacctctggggccagggaaccctggtcaccgtctcgagc

SEQ ID NO: 14 - protein, 122 aa, synthetic construct
  REGION 1..122: note = synthesized
EVQLLESGGGLVQPGGSLRLSCAASGFSFSSGYDMCWVRQAPGKGLEWIACIAAGSAGITYDANWAKGRFTISRDNSKNTLYLQMNSLRAEDTAVYYCARSAFSFDYAMDLWGQGTLVTVSS

SEQ ID NO: 15 - DNA, 330 bp, synthetic construct
  misc_feature 1..330: note = synthesized
  source 1..330: mol_type = other DNA
gacatccagatgacccagtctccttccaccctgtctgcatctgtaggagacagagtcaccatcacttgccaggccagtcagagcattagttcccacttaaactggtatcagcagaaaccagggaaagcccctaagctcctgatctataaggcatccactctggcatctggggtcccatcaaggttcagcggcagtggatctgggacagaatttactctcaccatcagcagcctgcagcctgatgattttgcaacttattactgccaacagggttatagttggggtaatgttgataatgttttcggcggagggaccaaggtggagatcaaa

SEQ ID NO: 16 - protein, 110 aa, synthetic construct
  REGION 1..110: note = synthesized
DIQMTQSPSTLSASVGDRVTITCQASQSISSHLNWYQQKPGKAPKLLIYKASTLASGVPSRFSGSGSGTEFTLTISSLQPDDFATYYCQQGYSWGNVDNVFGGGTKVEIK

SEQ ID NO: 17 - DNA, 345 bp, synthetic construct
  misc_feature 1..345: note = synthesized
  source 1..345: mol_type = other DNA
gaggtgcagctggtggagtctgggggaggcttggtccagcctggggggtccctgagactctcctgtacagcctctggattctccctaagtagctatgcaatgagctgggtccgccaggctccagggaaggggctggagtacatcggctacattggtgatactactggcatagcctacgcgagctgggcgaatggcagattcaccatctccaaagacaataccaagaacacggtggatcttcaaatgaacagcctgagagccgaggacacggctgtgtattactgtgcgagaggctggtcctacttagacatctggggccaagggaccctggtcaccgtctcgagc

SEQ ID NO: 18 - protein, 115 aa, synthetic construct
  REGION 1..115: note = synthesized
EVQLVESGGGLVQPGGSLRLSCTASGFSLSSYAMSWVRQAPGKGLEYIGYIGDTTGIAYASWANGRFTISKDNTKNTVDLQMNSLRAEDTAVYYCARGWSYLDIWGQGTLVTVSS

SEQ ID NO: 19 - DNA, 327 bp, synthetic construct
  misc_feature 1..327: note = synthesized
  source 1..327: mol_type = other DNA
gcccttgtgatgacccagtctccatcctccctgtctgcatctgtaggagacagagtcaccatcacttgccaggccagtcagaacatttacagcaatttagcctggtatcagcagaaaccagggaaagttcctaagctcctgatctatcaggcctccactctggcatctggggtcccatctcggttcagtggcagtggatatgggacagatttcactctcaccatcagcagcctgcagcctgaagatgttgcaacttattactgtcaaggcggttattatagtgctgcccttaatactttcggcggagggaccaaggtggagatcaaa

SEQ ID NO: 20 - protein, 109 aa, synthetic construct
  REGION 1..109: note = synthesized
ALVMTQSPSSLSASVGDRVTITCQASQNIYSNLAWYQQKPGKVPKLLIYQASTLASGVPSRFSGSGYGTDFTLTISSLQPEDVATYYCQGGYYSAALNTFGGGTKVEIK

SEQ ID NO: 21 - DNA, 360 bp, synthetic construct
  misc_feature 1..360: note = synthesized
  source 1..360: mol_type = other DNA
cagtcgctggtggagtctgggggaggcttggtacagcctggggggtccctgagactctcctgtgcagcctctggattctccttcagtagcaactactggatatgctgggtccgccaggctccagggaaggggctggagtggatcgcatgcatttatgttggtagtagtggtgacacttactacgcgagctccgcgaaaggccggttcaccatctccagagacaattccaagaacacgctgtatctgcaaatgaacagcctgagagccgaggacacggccgtatattactgtgcgagagatagtagtagttattatatgtttaacttgtggggccagggaaccctggtcaccgtctcgagc

SEQ ID NO: 22 - protein, 120 aa, synthetic construct
  REGION 1..120: note = synthesized
QSLVESGGGLVQPGGSLRLSCAASGFSFSSNYWICWVRQAPGKGLEWIACIYVGSSGDTYYASSAKGRFTISRDNSKNTLYLQMNSLRAEDTAVYYCARDSSSYYMFNLWGQGTLVTVSS

SEQ ID NO: 23 - DNA, 336 bp, synthetic construct
  misc_feature 1..336: note = synthesized
  source 1..336: mol_type = other DNA
gcccttgtgatgacccagtctccttccaccctgtctgcatctgtaggagacagagtcaccatcaattgccaggccagtgaggacattgatacctatttagcctggtatcagcagaaaccagggaaagcccctaagctcctgatcttttatgcatccgatctggcatctggggtcccatcaaggttcagcggcagtggatctgggacagaattcactctcaccatcagcagcctgcagcctgatgattttgcaacttattactgccaaggcggttactatactagtagtgctgatacgaggggtgctttcggcggagggaccaaggtggagatcaaa

SEQ ID NO: 24 - protein, 112 aa, synthetic construct
  REGION 1..112: note = synthesized
ALVMTQSPSTLSASVGDRVTINCQASEDIDTYLAWYQQKPGKAPKLLIFYASDLASGVPSRFSGSGSGTEFTLTISSLQPDDFATYYCQGGYYTSSADTRGAFGGGTKVEIK

SEQ ID NO: 25 - DNA, 345 bp, synthetic construct
  misc_feature 1..345: note = synthesized
  source 1..345: mol_type = other DNA
cggtcgctggtggagtctgggggaggcttggtccagcctggggggtccctgagactctcctgtacagcctctggattcaccatcagtagctaccacatgcagtgggtccgccaggctccagggaaggggctggagtacatcggaaccattagtagtggtggtaatgtatactacgcgagctccgcgagaggcagattcaccatctccagaccctcgtccaagaacacggtggatcttcaaatgaacagcctgagagccgaggacacggctgtgtattactgtgcgagagactctggttatagtgatcctatgtggggccagggaaccctggtcaccgtctcgagc

SEQ ID NO: 26 - protein, 115 aa, synthetic construct
  REGION 1..115: note = synthesized
RSLVESGGGLVQPGGSLRLSCTASGFTISSYHMQWVRQAPGKGLEYIGTISSGGNVYYASSARGRFTISRPSSKNTVDLQMNSLRAEDTAVYYCARDSGYSDPMWGQGTLVTVSS

SEQ ID NO: 27 - DNA, 333 bp, synthetic construct
  misc_feature 1..333: note = synthesized
  source 1..333: mol_type = other DNA
gacgttgtgatgacccagtctccatcttccgtgtctgcatctgtaggagacagagtcaccatcacctgtcaggccagtcagaacattaggacttacttatcctggtatcagcagaaaccagggaaagcccctaagctcctgatctatgctgcagccaatctggcatctggggtcccatcaaggttcagcggcagtggatctgggacagatttcactctcaccatcagcgacctggagcctggcgatgctgcaacttactattgtcagtctacctatcttggtactgattatgttggcggtgctttcggcggagggaccaaggtggagatcaaa

SEQ ID NO: 28 - protein, 111 aa, synthetic construct
  REGION 1..111: note = synthesized
DVVMTQSPSSVSASVGDRVTITCQASQNIRTYLSWYQQKPGKAPKLLIYAAANLASGVPSRFSGSGSGTDFTLTISDLEPGDAATYYCQSTYLGTDYVGGAFGGGTKVEIK

SEQ ID NO: 29 - DNA, 345 bp, synthetic construct
  misc_feature 1..345: note = synthesized
  source 1..345: mol_type = other DNA
gaggtgcagctgttggagtctgggggaggcttggtacagcctggggggtccctgagactctcctgtgcagcctctggaatcgacttcagtaggagatactacatgtgctgggtccgccaggctccagggaaggggctggagtggatcgcatgcatatatactggtagccgcgatactcctcactacgcgagctccgcgaaaggccggttcaccatctccagagacaattccaagaacacgctgtatctgcaaatgaacagcctgagagccgaggacacggccgtatattactgtgcgagagaaggtagcctgtggggccagggaaccctggtcaccgtctcgagc

SEQ ID NO: 30 - protein, 115 aa, synthetic construct
  REGION 1..115: note = synthesized
EVQLLESGGGLVQPGGSLRLSCAASGIDFSRRYYMCWVRQAPGKGLEWIACIYTGSRDTPHYASSAKGRFTISRDNSKNTLYLQMNSLRAEDTAVYYCAREGSLWGQGTLVTVSS

SEQ ID NO: 31 - DNA, 333 bp, synthetic construct
  misc_feature 1..333: note = synthesized
  source 1..333: mol_type = other DNA
gacatccagatgacccagtctccttccaccctgtctgcatctgtaggagacagagtcaccatcacttgccagtccagtcagagtgtttatagtaactggttctcctggtatcagcagaaaccagggaaagcccctaagctcctgatctattctgcatccactctggcatctggggtcccatcaaggttcagcggcagtggatctgggacagaattcactctcaccatcagcagcctgcagcctgatgattttgcaacttattactgcgcaggcggttacaatactgttattgatacttttgctttcggcggagggaccaaggtggagatcaaa

SEQ ID NO: 32 - protein, 111 aa, synthetic construct
  REGION 1..111: note = synthesized
DIQMTQSPSTLSASVGDRVTITCQSSQSVYSNWFSWYQQKPGKAPKLLIYSASTLASGVPSRFSGSGSGTEFTLTISSLQPDDFATYYCAGGYNTVIDTFAFGGGTKVEIK

SEQ ID NO: 33 - DNA, 357 bp, synthetic construct
  misc_feature 1..357: note = synthesized
  source 1..357: mol_type = other DNA
cagtcgctggtggagtctgggggaggcttggtccagcctggggggtccctgagactctcctgtactgcctctggattctccctcagtaggtactacatgacctgggtccgccaggctccagggaaggggctggagtggatcggaaccatttatactagtggtagtacatggtacgcgagctggacaaaaggcagattcaccatctccaaagacaataccaagaacacggtggatcttcaaatgaacagcctgagagccgaggacacggctgtgtattactgtgcgagatcctattatggcggtgataagactggtttaggcatctggggccagggaactctggttaccgtctcttca

SEQ ID NO: 34 - protein, 119 aa, synthetic construct
  REGION 1..119: note = synthesized
QSLVESGGGLVQPGGSLRLSCTASGFSLSRYYMTWVRQAPGKGLEWIGTIYTSGSTWYASWTKGRFTISKDNTKNTVDLQMNSLRAEDTAVYYCARSYYGGDKTGLGIWGQGTLVTVSS

SEQ ID NO: 35 - DNA, 339 bp, synthetic construct
  misc_feature 1..339: note = synthesized
  source 1..339: mol_type = other DNA
gacatccagatgacccagtctccttccaccctgtctgcatctgtaggagacagagtcaccatcacttgccaggccagtcagagcattgatagttggttatcctggtatcagcagaaaccagggaaagcccctaagctcctgatctatcaggcatccactctggcatctggggtcccatcaaggttcagcggcagtggatctgggacagagttcactctcaccatcagcagcctgcagcctgatgattttgcaacttattactgccaatctgcttatggtgttagtggtactagtagttatttatatactttcggcggagggaccaaggtggagatcaaa

SEQ ID NO: 36 - protein, 113 aa, synthetic construct
  REGION 1..113: note = synthesized
DIQMTQSPSTLSASVGDRVTITCQASQSIDSWLSWYQQKPGKAPKLLIYQASTLASGVPSRFSGSGSGTEFTLTISSLQPDDFATYYCQSAYGVSGTSSYLYTFGGGTKVEIK

SEQ ID NO: 37 - DNA, 366 bp, synthetic construct
  misc_feature 1..366: note = synthesized
  source 1..366: mol_type = other DNA
gaggtgcagctgttggagtctgggggaggcttggtacagcctggggggtccctgagactctcctgtgcagcctctggattcaccatcagtcgctaccacatgacttgggtccgccaggctccagggaaggggctggagtggatcggacatatttatgttaataatgatgacacagactacgcgagctccgcgaaaggccggttcaccatctccagagacaattccaagaacacgctgtatctgcaaatgaacagcctgagagccgaggacacggccacctatttctgtgcgagattggatgttggtggtggtggtgcttatattggggacatctggggccagggaactctggttaccgtctcttca

SEQ ID NO: 38 - protein, 122 aa, synthetic construct
  REGION 1..122: note = synthesized
EVQLLESGGGLVQPGGSLRLSCAASGFTISRYHMTWVRQAPGKGLEWIGHIYVNNDDTDYASSAKGRFTISRDNSKNTLYLQMNSLRAEDTATYFCARLDVGGGGAYIGDIWGQGTLVTVSS

SEQ ID NO: 39 - DNA, 339 bp, synthetic construct
  misc_feature 1..339: note = synthesized
  source 1..339: mol_type = other DNA
gacatccagatgacccagtctccatcctccctgtctgcatctgtaggagacagagtcaccatcacttgccagtccagtcagagtgtttataacaacaacgacttagcctggtatcagcagaaaccagggaaagttcctaagctcctgatctattatgcttccactctggcatctggggtcccatctcggttcagtggcagtggatctgggacagatttcactctcaccatcagcagcctgcagcctgaagatgttgcaacttattactgtgcaggcggttatgatacggatggtcttgatacgtttgctttcggcggagggaccaaggtggagatcaaa

SEQ ID NO: 40 - protein, 113 aa, synthetic construct
  REGION 1..113: note = synthesized
DIQMTQSPSSLSASVGDRVTITCQSSQSVYNNNDLAWYQQKPGKVPKLLIYYASTLASGVPSRFSGSGSGTDFTLTISSLQPEDVATYYCAGGYDTDGLDTFAFGGGTKVEIK

SEQ ID NO: 41 - DNA, 345 bp, synthetic construct
  misc_feature 1..345: note = synthesized
  source 1..345: mol_type = other DNA
gaggtgcagctggtggagtctgggggaggcttggtccagcctggggggtccctgagactctcctgtactgcctctggattctccctcagtagctatgcaatgagctgggtccgccaggctccagggagggggctggagtggatcggaatcatttatgctagtggtagcacatactacgcgagctcggcgaaaggcagattcaccatctccaaagacaataccaagaacacggtggatcttcaaatgaacagcctgagagccgaggacacggctgtgtattactgtgcgagaatttatgacggcatggacctctggggccagggaactctggttaccgtctcttca

SEQ ID NO: 42 - protein, 115 aa, synthetic construct
  REGION 1..115: note = synthesized
EVQLVESGGGLVQPGGSLRLSCTASGFSLSSYAMSWVRQAPGRGLEWIGIIYASGSTYYASSAKGRFTISKDNTKNTVDLQMNSLRAEDTAVYYCARIYDGMDLWGQGTLVTVSS

SEQ ID NO: 43 - DNA, 321 bp, synthetic construct
  misc_feature 1..321: note = synthesized
  source 1..321: mol_type = other DNA
gacatccagatgacccagtctccatcctccctgtctgcatctgtaggagacagagtcaccatcaattgccaggccagtcagaacatttacagctacttatcctggtatcagcagaaaccagggaaagttcctaagcgcctgatctatctggcatctactctggcatctggggtcccatctcggttcagtggcagtggatctgggacagattacactctcaccatcagcagcctgcagcctgaagatgttgcaacttattactgtcaaagcaattataacggtaattatggtttcggcggagggaccaaggtggagatcaaa

SEQ ID NO: 44 - protein, 107 aa, synthetic construct
  REGION 1..107: note = synthesized
DIQMTQSPSSLSASVGDRVTINCQASQNIYSYLSWYQQKPGKVPKRLIYLASTLASGVPSRFSGSGSGTDYTLTISSLQPEDVATYYCQSNYNGNYGFGGGTKVEIK

SEQ ID NO: 45 - DNA, 360 bp, synthetic construct
  misc_feature 1..360: note = synthesized
  source 1..360: mol_type = other DNA
gaggtgcagctggtggagtctgggggaggcttggtccagcctggggggtccctgagactctcctgtgcagcctctggattctccctcaataactactggatgagctgggtccgccaggctccagggaaggggctggagtggatcggaaccattagtagtggtgcgtatacatggttcgccacctgggcgacaggcagattcaccatctccagagacaattccaagaacacgctgtatcttcaaatgaacagcctgagagccgaggacacggctgtgtattactgtgcgagatattcttctactactgattggacctactttaacatctggggccagggaactctggttaccgtctcttca

SEQ ID NO: 46 - protein, 120 aa, synthetic construct
  REGION 1..120: note = synthesized
EVQLVESGGGLVQPGGSLRLSCAASGFSLNNYWMSWVRQAPGKGLEWIGTISSGAYTWFATWATGRFTISRDNSKNTLYLQMNSLRAEDTAVYYCARYSSTTDWTYFNIWGQGTLVTVSS

SEQ ID NO: 47 - DNA, 324 bp, synthetic construct
  misc_feature 1..324: note = synthesized
  source 1..324: mol_type = other DNA
gacatccagatgacccagtctccttccaccctgtctgcatctgtaggagacagagtcaccatcacttgccaggccagtcagagcattaataactacttagcctggtatcagcagaaaccagggaaagcccctaagctcctgatctatagggcatccactctggaatctggggtcccatcaaggttcagcggcagtggatctgggacagaattcactctcaccatcagcagcctgcagcctgatgattttgcaacttattactgccaaagctataatggtgttggtaggactgctttcggcggagggaccaaggtggagatcaaa

SEQ ID NO: 48 - protein, 108 aa, synthetic construct
  REGION 1..108: note = synthesized
DIQMTQSPSTLSASVGDRVTITCQASQSINNYLAWYQQKPGKAPKLLIYRASTLESGVPSRFSGSGSGTEFTLTISSLQPDDFATYYCQSYNGVGRTAFGGGTKVEIK

SEQ ID NO: 49 - DNA, 348 bp, synthetic construct
  misc_feature 1..348: note = synthesized
  source 1..348: mol_type = other DNA
gatgtgcagcttcaggagtcgggacctagcctggtgaaaccttctcagtctctgtccctcacctgcactgtcactggctactcaatcaccagtgattttgcctggaactggattcggcagtttccaggaaacaagctggagtggatgggctacataagttatagtggtaacactaggtacaacccatctctcaaaagtcgaatctctatcactcgcgacacatccaagaaccaattcttcctgcagttgaactctgtgactattgaggacacagccacatattactgtgtaacggcgggacgcgggtttccttattggggccaagggactctggtcactgtctctgca

SEQ ID NO: 50 - protein, 116 aa, synthetic construct
  REGION 1..116: note = synthesized
DVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSA

SEQ ID NO: 51 - DNA, 321 bp, synthetic construct
  misc_feature 1..321: note = synthesized
  source 1..321: mol_type = other DNA
gacatcctgatgacccaatctccatcctccatgtctgtatctctgggagacacagtcagcatcacttgccattcaagtcaggacattaacagtaatatagggtggttgcagcagagaccagggaaatcatttaagggcctgatctatcatggaaccaacttggacgatgaagttccatcaaggttcagtggcagtggatctggagccgattattctctcaccatcagcagcctggaatctgaagattttgcagactattactgtgtacagtatgctcagtttccgtggacgttcggtggaggcaccaagctggaaatcaaa

SEQ ID NO: 52 - protein, 107 aa, synthetic construct
  REGION 1..107: note = synthesized
DILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIK

SEQ ID NO: 53 - DNA, 363 bp, synthetic construct
  misc_feature 1..363: note = synthesized
  source 1..363: mol_type = other DNA
gaggtgcagctggtgcagtctggagcagaggtgaagaaaccaggagagtctctgaagatctcctgtaagggttctggatacagctttagcagttcatggatcggctgggtgcgccaggcacctgggaaaggcctggaatggatggggatcatctatcctgatgactctgataccagatacagtccatccttccaaggccaggtcaccatctcagccgacaagtccatcaggactgcctacctgcagtggagtagcctgaaggcctcggacaccgctatgtattactgtgcgagacatgttactatgatttggggagttattattgacttctggggccagggaaccctggtcaccgtctcctca

SEQ ID NO: 54 - protein, 121 aa, synthetic construct
  REGION 1..121: note = synthesized
EVQLVQSGAEVKKPGESLKISCKGSGYSFSSSWIGWVRQAPGKGLEWMGIIYPDDSDTRYSPSFQGQVTISADKSIRTAYLQWSSLKASDTAMYYCARHVTMIWGVIIDFWGQGTLVTVSS

SEQ ID NO: 55 - DNA, 321 bp, synthetic construct
  misc_feature 1..321: note = synthesized
  source 1..321: mol_type = other DNA
gccatccagttgacccagtctccatcctccctgtctgcatctgtaggagacagagtcaccatcacttgccgggcaagtcagggcattagcagtgctttagcctggtatcagcagaaaccagggaaagctcctaagctcctgatctatgatgcctccagtttggaaagtggggtcccatcaaggttcagcggcagtggatctgggacagatttcactctcaccatcagcagcctgcagcctgaagattttgcaacttattactgtcaacagtttaatagttacccattcactttcggccctgggaccaaagtggatatcaaa

SEQ ID NO: 56 - protein, 107 aa, synthetic construct
  REGION 1..107: note = synthesized
AIQLTQSPSSLSASVGDRVTITCRASQGISSALAWYQQKPGKAPKLLIYDASSLESGVPSRFSGSGSGTDFTLTISSLQPEDFATYYCQQFNSYPFTFGPGTKVDIK

SEQ ID NO: 57 - DNA, 354 bp, synthetic construct
  misc_feature 1..354: note = synthesized
  source 1..354: mol_type = other DNA
gaggtgaagctggatgagactggaggaggcttggtgcaacctgggaggcccatgaaactctcctgtgttgcctctggattcacttttagtgactactggatgaactgggtccgccagtctccagagaaaggactggagtgggtagcacaaattagaaacaaaccttataattatgaaacatattattcagattctgtgaaaggcagattcaccatctcaagagatgattccaaaagtagtgtctacctgcaaatgaacaacttaagagttgaagacatgggtatctattactgtacgggttcttactatggtatggactactggggtcaaggaacctcagtcaccgtctcctca

SEQ ID NO: 58 - protein, 118 aa, synthetic construct
  REGION 1..118: note = synthesized
EVKLDETGGGLVQPGRPMKLSCVASGFTFSDYWMNWVRQSPEKGLEWVAQIRNKPYNYETYYSDSVKGRFTISRDDSKSSVYLQMNNLRVEDMGIYYCTGSYYGMDYWGQGTSVTVSS

SEQ ID NO: 59 - DNA, 336 bp, synthetic construct
  misc_feature 1..336: note = synthesized
  source 1..336: mol_type = other DNA
gatgtcgtgatgacccaaactccactctccctgcctgtcagtcttggagatcaagcctccatctcttgcagatctagtcagagccttgtacacagtaatggaaacacctatttacgttggtacctgcagaagccaggccagtctccaaaggtcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgtggacgttcggtggaggcaccaagctggaaatcaaa

SEQ ID NO: 60 - protein, 112 aa, synthetic construct
  REGION 1..112: note = synthesized
DVVMTQTPLSLPVSLGDQASISCRSSQSLVHSNGNTYLRWYLQKPGQSPKVLIYKVSNRFSGVPDRFSGSGSGTDFTLKISRVEAEDLGVYFCSQSTHVPWTFGGGTKLEIK

SEQ ID NO: 61 - DNA, 987 bp, synthetic construct
  misc_feature 1..987: note = synthesized
  source 1..987: mol_type = other DNA
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggt

SEQ ID NO: 62 - protein, 329 aa, synthetic construct
  REGION 1..329: note = synthesized
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 63 - DNA, 321 bp, synthetic construct
  misc_feature 1..321: note = synthesized
  source 1..321: mol_type = other DNA
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 64 - protein, 107 aa, synthetic construct [sequence omitted]

SEQ ID NO: 65 - DNA, 3681 bp, synthetic construct
  misc_feature 1..3681: note = synthesized
  source 1..3681: mol_type = other DNA
gacatccagatgacccagtctccttccaccctgtctgcatctgtaggagacagagtcaccatcacttgccagtccagtcagagtgtttatagtaactggttctcctggtatcagcagaaaccagggaaagcccctaagctcctgatctattctgcatccactctggcatctggggtcccatcaaggttcagcggcagtggatctgggacagaattcactctcaccatcagcagcctgcagcctgatgattttgcaacttattactgcgcaggcggttacaatactgttattgatacttttgctttcggcggagggaccaaggtggagatcaaaggcggtggcggtagtgggggaggcggttctggcggcggagggtccggcggtggaggatcagaggtgcagctgttggagtctgggggaggcttggtacagcctggggggtccctgagactctcctgtgcagcctctggaatcgacttcagtaggagatactacatgtgctgggtccgccaggctccagggaaggggctggagtggatcgcatgcatatatactggtagccgcgatactcctcactacgcgagctccgcgaaaggccggttcaccatctccagagacaattccaagaacacgctgtatctgcaaatgaacagcctgagagccgaggacacggccgtatattactgtgcgagagaaggtagcctgtggggccagggaaccctggtcaccgtctcgagcggcggtggagggtccggcggtggtggatcccagtcggtggaggagtctgggggaggcttggtccagcctggggggtccctgagactctcctgtacagcctctggaatcgaccttaatacctacgacatgatctgggtccgccaggctccaggcaaggggctagagtgggttggaatcattacttatagtggtagtagatactacgcgaactgggcgaaaggccgattcaccatctccaaagacaataccaagaacacggtgtatctgcaaatgaacagcctgagagctgaggacacggctgtgtattactgtgccagagattatatgagtggttcccacttgtggggccagggaaccctggtcaccgtctctagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagccgcgggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcgcggtctccaacaaagccctcccagcccccatcgagaaaaccatctccaaagccaaagggcagccccgagaaccacaggtgtataccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtggcggtggagggtccggcggtggtggatccgaggtgcagctgttggagtctgggggaggcttggtacagcctggggggtccctgagactctcctgtgcagcctctggattcaccatcagtcgctaccacatgacttgggtccgccaggctccagggaaggggctggagtggatcggacatatttatgttaataatgatgacacagactacgcgagctccgcgaaaggccggttcaccatctccagagacaattccaagaacacgctgtatctgcaaatgaacagcctgagagccgaggacacggccacctatttctgtgcgagattggatgttggtggtggtggtgcttatattggggacatctggggccagggaactctggttaccgtctcttcaggcggtggcggtagtgggggaggcggttctggcggcggagggtccggcggtggaggatcagacatccagatgacccagtctccatcctccctgtctgcatctgtaggagacagagtcaccatcacttgccagtccagtcagagtgtttataacaacaacgacttagcctggtatcagcagaaaccagggaaagttcctaagctcctgatctattatgcttccactctggcatctggggtcccatctcggttcagtggcagtggatctgggacagatttcactctcaccatcagcagcctgcagcctgaagatgttgcaacttattactgtgcaggcggttatgatacggatggtcttgatacgtttgctttcggcggagggaccaaggtggagatcaaaggcggtggagggtccggcggtggtggatccgaggtgcagctggtggagtctgggggaggcttggtccagcctggggggtccctgagactctcctgtgcagcctctggattcaccatcagtaccaatgcaatgagctgggtccgccaggctccagggaaggggctggagtggatcggagtcattactggtcgtgatatcacatactacgcgagctgggcgaaaggcagattcaccatctccagagacaattccaagaacacgctgtatcttcaaatgaacagcctgagagccgaggacacggctgtgtattactgtgcgcgcgacggtggatcatctgctattactagtaacaacatttggggccaaggaactctggtcaccgtttcttcaggcggtggcggtagtgggggaggcggttctggcggcggagggtccggcggtggaggatcagacgtcgtgatgacccagtctccttccaccctgtctgcatctgtaggagacagagtcaccatcaattgccaagccagtgagagcattagcagttggttagcctggtatcagcagaaaccagggaaagcccctaagctcctgatctatgaagcatccaaactggcatctggggtcccatcaaggttcagcggcagtggatctgggacagagttcactctcaccatcagcagcctgcagcctgatgattttgcaacttattactgccaaggctatttttattttattagtcgtacttatgtaaattctttcggcggagggaccaaggtggagatcaaa

SEQ ID NO: 66 - protein, 1227 aa, synthetic construct
  REGION 1..1227: note = synthesized
DIQMTQSPSTLSASVGDRVTITCQSSQSVYSNWFSWYQQKPGKAPKLLIYSASTLASGVPSRFSGSGSGTEFTLTISSLQPDDFATYYCAGGYNTVIDTFAFGGGTKVEIKGGGGSGGGGSGGGGSGGGGSEVQLLESGGGLVQPGGSLRLSCAASGIDFSRRYYMCWVRQAPGKGLEWIACIYTGSRDTPHYASSAKGRFTISRDNSKNTLYLQMNSLRAEDTAVYYCAREGSLWGQGTLVTVSSGGGGSGGGGSQSVEESGGGLVQPGGSLRLSCTASGIDLNTYDMIWVRQAPGKGLEWVGIITYSGSRYYANWAKGRFTISKDNTKNTVYLQMNSLRAEDTAVYYCARDYMSGSHLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEVQLLESGGGLVQPGGSLRLSCAASGFTISRYHMTWVRQAPGKGLEWIGHIYVNNDDTDYASSAKGRFTISRDNSKNTLYLQMNSLRAEDTATYFCARLDVGGGGAYIGDIWGQGTLVTVSSGGGGSGGGGSGGGGSGGGGSDIQMTQSPSSLSASVGDRVTITCQSSQSVYNNNDLAWYQQKPGKVPKLLIYYASTLASGVPSRFSGSGSGTDFTLTISSLQPEDVATYYCAGGYDTDGLDTFAFGGGTKVEIKGGGGSGGGGSEVQLVESGGGLVQPGGSLRLSCAASGFTISTNAMSWVRQAPGKGLEWIGVITGRDITYYASWAKGRFTISRDNSKNTLYLQMNSLRAEDTAVYYCARDGGSSAITSNNIWGQGTLVTVSSGGGGSGGGGSGGGGSGGGGSDVVMTQSPSTLSASVGDRVTINCQASESISSWLAWYQQKPGKAPKLLIYEASKLASGVPSRFSGSGSGTEFTLTISSLQPDDFATYYCQGYFYFISRTYVNSFGGGTKVEIK

SEQ ID NO: 67 - DNA, 651 bp, synthetic construct
  misc_feature 1..651: note = synthesized
  source 1..651: mol_type = other DNA
gcctatgatatgacccagtctccatcttccgtgtctgcatctgtaggagacagagtcaccatcaagtgtcaggccagtgaggacatttatagcttcttggcctggtatcagcagaaaccagggaaagcccctaagctcctgatccattctgcatcctctctggcatctggggtcccatcaaggttcagcggcagtggatctgggacagatttcactctcaccatcagcagcctgcagcctgaagattttgcaacttactattgtcaacagggttatggtaaaaataatgttgataatgctttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 68 - protein, 217 aa, synthetic construct
  REGION 1..217: note = synthesized
AYDMTQSPSSVSASVGDRVTIKCQASEDIYSFLAWYQQKPGKAPKLLIHSASSLASGVPSRFSGSGSGTDFTLTISSLQPEDFATYYCQQGYGKNNVDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0279333 A1
(43) Pub. Date: Aug. 22, 2024

(54) MULTI-SPECIFIC ANTIBODIES AND METHODS OF MAKING AND USING THEREOF

(71) Applicants: SYSTIMMUNE, INC., Redmond, WA (US); BAILI-BIO (CHENGDU) PHARMACEUTICAL CO., LTD., Chengdu (CN)

(72) Inventors: Yi ZHU, Chengdu (CN); Ole OLSEN, Everett, WA (US); Dong XIA, Redmond, WA (US); David JELLYMAN, Duvall, WA (US); Katrina BYKOVA, Seattle, WA (US); Anne-Marie K. ROUSSEAU, Seattle, WA (US); Bill BRADY, Bothell, WA (US); Blair RENSHAW, Renton, WA (US); Brian KOVACEVICH, Snohomish, WA (US); Yu LIANG, Redmond, WA (US); Zeren GAO, Redmond, WA (US)

(21) Appl. No.: 18/459,019

(22) Filed: Aug. 30, 2023

Related U.S. Application Data
(63) Continuation of application No. 16/615,122, filed on Nov. 19, 2019, now Pat. No. 11,787,863.

(51) Int. Cl. C07K 16/28; A61P 35/02

(52) U.S. Cl. CPC C07K 16/2809; A61P 35/02; C07K 16/2803; C07K 16/2827; C07K 16/2863; C07K 16/2878; C07K 2317/31; C07K 2317/52; C07K 2317/55; C07K 2317/622; C07K 2317/92

(57) ABSTRACT

The disclosure provides a tetra-specific antibody monomer having a N-terminal and a C-terminal, comprising in tandem from the N-terminal to the C-terminal, a first scFv domain at the N-terminal, a Fab domain, a Fc domain, a second scFv domain, and a third scFv at the C-terminal, wherein the first scFv domain, the Fab domain, the second scFv domain, and the third scFv domain each has a binding specificity against a different antigen. In one embodiment, the antigen is a tumor antigen, an immune signaling antigen, or a combination thereof. Multi-specific antibodies comprising the disclosed tetra-specific antibodies are also provided.

### CROSS REFERENCE TO RELATED APPLICATIONS

This application claims the benefit of U.S. Provisional Patent Application No. 62/524,557 filed Jun. 25, 2017, which application is expressly incorporated herein by reference in its entirety.

### TECHNICAL FIELD

The present disclosure generally relates to the technical field of biologic therapeutics, and more particularly relates to making and using multi-specific antibodies.

### BACKGROUND

Cancer cells develop various strategies to evade the immune system. One of the underlying mechanisms for the immune escape is the reduced recognition of cancer cells by the immune system. Defective presentation of cancer specific antigens or lack of thereof results in immune tolerance and cancer progression. In the presence of effective immune recognition tumors use other mechanisms to avoid elimination by the immune system. Immunocompetent tumors create suppressive microenvironment to downregulate the immune response. Multiple players are involved in shaping the suppressive tumor microenvironment, including tumor cells, regulatory T cells, Myeloid-Derived Suppressor cells, stromal cells, and other cell types. The suppression of immune response may be executed in a cell contact-dependent format as well as in and a contact-independent manner, via secretion of immunosuppressive cytokines or elimination of essential survival factors from the local environment. Cell contact-dependent suppression relies on molecules expressed on the cell surface, e.g. Programmed Death Ligand 1 (PD-L1), T-lymphocyte-associated protein 4 (CTLA-4) and others [Dunn, et al., 2004, Immunity, 21(2): 137-48; Adachi & Tamada, 2015, Cancer Sci., 106(8): 945-50].

As the mechanisms by which tumors evade recognition by the immune system continue to be better understood new treatment modalities that target these mechanisms have recently emerged. On Mar. 25, 2011, the U. S. Food and Drug Administration (FDA) approved ipilimumab (anti-CTLA-4 monoclonal antibody, YERVOY®, Bristol-Myers Squibb) injection for the treatment of unresectable or metastatic melanoma. YERVOYS binds to cytotoxic T-lymphocyte-associated protein 4 (CTLA-4) expressed on activated T cells and blocks the interaction of CTLA-4 with CD80/86 on antigen-presenting cells thereby blocking the negative or inhibitory signal delivered into the T cell through CTLA-4 resulting in re-activation of the antigen-specific T cell leading to, in many patients, eradication of the tumor. A few years later in 2014 the FDA approved KEYTRUDA® (anti-PD1 monoclonal antibody, Pembrolizumab, Merck) and OPDIVO® (anti-PD1 monoclonal antibody, Nivolumab, Bristol-Myers Squibb) for treatment of advanced melanoma. These monoclonal antibodies bind to PD-1 which is expressed on activated and/or exhausted T cells and block the interaction of PD-1 with PD-L1 expressed on tumors thereby eliminating the inhibitory signal through PD-1 into the T cell resulting in re-activation of the antigen-specific T cell leading to again, in many patients, eradication of the tumor. Since then additional clinical trials have been performed comparing the single monoclonal antibody Yervoy® to the combination of the monoclonal antibodies YERVOY® and OPDIVO® in the treatment of advanced melanoma which showed improvement in overall survival and progression-free survival in the patients treated with the combination of antibodies. (Hodi et al., 2016, Lancet Oncol. 17(11):1558-1568, Hellman et al., 2018, Cancer Cell 33(5): 853-861). However, as many clinical trials have shown a great benefit of treating cancer patients with monoclonal antibodies that are specific for one or more immune checkpoint molecules data has emerged that only those patients with a high mutational burden that generates a novel T cell epitope(s) which is recognized by antigen-specific T cells show a clinical response (Snyder et al., 2014, NEJM 371:2189-2199). Those patients that have a low tumor mutational load mostly do not show an objective clinical response (Snyder et al., 2014, NEJM 371:2189-2199, Hellman et al., 2018, Cancer Cell 33(5): 853-861).

In recent years, other groups have developed an alternate approach that does not require the presence of neoepitope presentation by antigen-presenting cells to activate T cells. One example is the development of a bi-specific antibody where the binding domain of an antibody which is specific for a tumor associated antigen, e.g., CD19, is linked to and antibody binding domain specific for CD3 on T cells thus creating a bi-specific T cell engager or BiTe molecule. In 2014, the FDA approved a bi-specific antibody called Blinatumomab (bispecific anti-CD3×CD19 monoclonal antibody, BLINCYTO®; Amgen) for the treatment of Precursor B-Cell Acute Lymphoblastic Leukemia. Blinatumomab links the scFv specific for CD19 expressed on leukemic cells with the scFv specific for CD3 expressed on T cells (Bejnjamin and Stein 2016, Ther Adv Hematol 7(3):142-146). However, despite an initial response rate of >50% in patients with relapsed or refractory ALL many patients are resistant to Blinatumomab therapy or relapse after successful treatment with Blinatumomab. Evidence is emerging that the resistant to Blinatumomab or who relapse after Blinatumomab treatment is attributable to the expression of immune checkpoint inhibitory molecules expressed on tumor cells such as PD-L1 that drives an inhibitory signal through PD-1 expressed on activated T cells (Feucht et al., 2016, Oncotarget 7(47): 76902-76919). In a case study of a patient who was resistant to therapy with Blinatumomab a second round of Blinatumomab therapy was performed but with the addition of a monoclonal antibody, pembrolizumab (KEYTRUDA®, Merck), which specific for PD-1 and blocks the interaction of T cell-expressed PD-1 with tumor cell expressed PD-L1 resulted in a dramatic response and reduction of tumor cells in the bone marrow from 45% to less than 5% in this one patient (Feucht et al., 2016, Oncotarget 7(47): 76902-76919). These results show that combining a bi-specific BiTe molecule with one or more monoclonal antibodies may significantly increase clinical activity compared to either agent alone.

### SUMMARY

The present disclosure provides, among others, tetra-specific antibody monomers, antibodies containing tetra-specific monomers, antigen-binding fragments thereof, multi-specific antibodies, immuno-conjugates comprising the disclosed antibodies, methods of making disclosed monomers, antigen-binding fragments, and antibodies, and methods of using the disclosed molecules for treating cancer.

In one aspect, the application provides tetra-specific antibody monomers. In one embodiment, the tetra-specific antibody monomer has a N-terminal and a C-terminal and include in tandem from the N-terminal to the C-terminal, a first scFv domain at the N-terminal, a Fab domain, a Fc domain, a second scFv domain, and a third scFv at the C-terminal. The first scFv domain, the Fab domain, the second scFv domain, and the third scFv domain each has a binding specificity against a different antigen.

In one embodiment, the antigen includes a tumor antigen, an immune signalling antigen, or a combination thereof. In one embodiment, the first scFv domain, the Fab domain, the second scFv domain, and the third scFv domain each has a binding specificity against a tumor antigen or an immune signalling antigen. In one embodiment, the first scFv domain has a binding specificity against a tumor antigen. In one embodiment, the first scFv domain has a binding specificity against an immune signalling antigen. In one embodiment, the Fab domain has a binding specificity against a tumor antigen. In one embodiment, the Fab domain has a binding specificity against an immune signalling antigen. In one embodiment, the second scFv domain has a binding specificity against a tumor antigen. In one embodiment, the second scFv domain has a binding specificity against an immune signalling antigen. In one embodiment, the third scFv domain has a binding specificity against a tumor antigen. In one embodiment, the third scFv domain has a binding specificity against a tumor antigen.

In one embodiment, the tetra-specific monomer includes the scFv domain, the Fab domain, the second scFv domain, and the third scFv domain each independently has a binding specificity against an antigen selected from ROR1, PD-L1, CD3, CD28, 4-1BB, CEA, HER2, EGFR VIII, EGFR, LMP1, LMP2A, Mesothelin, PSMA, EpCAM, glypimay-3, gpA33, GD2, TROP2, NKG2D, BCMA, CD19, CD20, CD33, CD123, CD22, CD30, PD-L1, PD1, OX40, 4-1BB, GITR, TIGIT, TIM-3, LAG-3, CTLA4, CD40, VISTA, ICOS, BTLA, LIGHT, HVEM, CSF1R, CD73, and CD39. In one embodiment, the scFv domain, the Fab domain, the second scFv domain, and the third scFv domain each independently has a binding specificity against tumor specific antigens including, but not limited to, ROR1, CEA, HER2, EGFR, EGFR VIII, LMP1, LMP2A, Mesothelin, PSMA, EpCAM, glypimay-3, gpA33, GD2, TROP2, BCMA, CD3, CD19, CD20, CD33, CD123, CD22, CD30, or immune checkpoint modulators including, without limitation, PD-L1, PD1, OX40, 4-1BB, GITR, TIGIT, TIM-3, LAG-3, CTLA4, CD40, VISTA, ICOS, BTLA, Light, HVEM, CD73, CD39, etc. In one embodiment, one set of scFv domain may specifically bind to an immune checkpoint modulators or a tumor antigen. In one embodiment, the scFv specific to CD3 may be on either C or N terminal of heave or light chains.

In one embodiment, the first scFv domain, the Fab domain, the second scFv domain, and the third scFv domain each independently has a binding specificity against an antigen selected from CD3, EGFRvIII, PD-L1, and 4-1BB. In one embodiment, the first scFv domain has a binding specificity against CD3. In one embodiment, the Fab domain has a binding specificity against EGFRvIII. In one embodiment, the second scFv domain has a binding specificity against PD-L1. In one embodiment, the third scFv domain has a binding specificity against 4-1BB. In one embodiment, the first scFv domain has a binding specificity against CD3, the Fab domain has a binding specificity against EGFRvIII, the second scFv domain has a binding specificity against PD-L1, and the third scFv domain has a binding specificity against 4-1BB.

Fc domain may be humanized. In one embodiment, the Fc domain is a human IgG1 Fc.

The scFv domain may include a linker linking the scFv domain to the heavy chain or light chain of the antibody. In one embodiment, the linker may include more than 10 amino acids. In one embodiment, the linker may include more than 15 amino acids long. In one embodiment, the linker may include less than 20 amino acids.

In one embodiment, the linker may comprises a gly-gly-gly-gly-ser (G4S)n linker, and n may be an integral between 1 to 20. For example, n may be 2, 4, or 6. In one embodiment, the first scFv domain, the second scFv domain, or the third scFv domain may comprise a gly-gly-gly-gly-ser (G4S)n linker, wherein n is 2 or 4.

In one embodiment, the application provides a tetra-specific antibody monomers having an amino acid sequence having a percentage homology to SEQ ID NO. 02, 04, 06, 08, 10, 12, 14, 16, 18, 20, 22, 24, 26, 28, 30, 32, 34, 36, 38, 40, 42, 44, 46, 48, 50, 52, 54, 56, 58, and 60. The percentage homology is not less than 70%, 80%, 90%, 95%, 98% or 99%.

The application further provides antigen-binding fragments. In one embodiment, the application provides scFv domains. In one embodiment, the scFv domain has an amino acid sequence having a percentage homology to SEQ ID NO. 02, 04, 06, 08, 10, 12, 14, 16, 18, 20, 22, 24, 26, 28, 30, 32, 34, 36, 38, 40, 42, 44, 46, 48, 50, 52, 54, 56, 58, and 60, wherein the percentage homology is not less than 70%, 80%, 90%, 95%, 98% or 99%. In one embodiment, the application provides Fab domains. In one embodiment, the Fab domain includes an amino acid sequence having a percentage homology to SEQ ID NO. 02, 04, 06, 08, 10, 12, 14, 16, 18, 20, 22, 24, 26, 28, 30, 32, 34, 36, 38, 40, 42, 44, 46, 48, 50, 52, 54, 56, 58 and 60, wherein the percentage homology is not less than 70%, 80%, 90%, 95%, 98% or 99%. The antigen-binding fragments disclosed herein may be used to construct the tetra-specific antibody monomers or multi-specific antibodies.

In one aspect, the application provides multi-specific antibodies. In one embodiment, the multi-specific antibody includes tetra-specific antibody monomers. In one embodiment, the multi-specific antibody includes two tetra-specific antibody monomers disclosed herein. As each tetra-specific antibody monomer has four antigen-binding domains, the multi-specific antibody disclosed may include 8 antigen-binding domains. In one embodiment, the antigen binding domains in such multi-specific antibody each independently has a binding specificity against a different antigen therefor providing an octa-specific antibody. In one embodiment, the multi-specific antibody is a penta-specific antibody. In one embodiment, the multi-specific antibody is a penta-specific antibody. In one embodiment, the multi-specific antibody is a penta-specific antibody a hexa-specific antibody. In one embodiment, the multi-specific antibody is a penta-specific antibody a hepta-specific antibody.

In one embodiment, the multi-specific antibody includes a dimer of a tetra-specific antibody monomer therefor providing a tetra-specific antibody. In one embodiment, the application provides an isolated, purified, or non-natural existing multi-specific antibodies. In one embodiment, the application provides a tetra-specific antibody having an amino acid sequence having a percentage homology to SEQ ID 66 and 68. The percentage homology is not less than 70%, 80%, 90%, 95%, 98% or 99%.

The application further provides isolated nucleic acid sequence encoding the tetra-specific antibody monomers, the multi-specific antibodies, or the antigen-binding fragments thereof. In one embodiment, the nucleic acid encodes an amino acid sequence having a percentage homology to the tetra-specific antibody monomer having a SEQ ID NO. 01, 03, 05, 07, 09, 11, 13, 15, 17, 19, 21, 23, 25, 27, 29, 31, 33, 35, 37, 39, 41, 43, 45, 47, 49, 51, 53, 55, 57, and 59. The percentage homology is not less than 70%, 80%, 90%, 95%, 98% or 99%.

The application further provides expression vectors and host cells comprising the nucleic acid sequences disclosed herein. In one embodiment, the host cell includes the expression vector. The host cell may be a prokaryotic cell or a eukaryotic cell.

The application further provides immuno-conjugates. In one embodiment, the immuno-conjugate includes a cytotoxic agent or an imaging agent linked to the multi-specific antibody disclosed herein through a linker.

The linker may be cleavable or non-cleavable. The linker may include a covalent bond such as an ester bond, an ether bond, an amid bond, a disulphide bond, an imide bond, a sulfone bond, a phosphate bond, a phosphorus ester bond, a peptide bond, or a combination thereof. In one embodiment, the linker comprises a hydrophobic poly(ethylene glycol) linker.

The cytotoxic agent may include a chemotherapeutic agent, a growth inhibitory agent, a cytotoxic agent from class of calicheamicin, an antimitotic agent, a toxin, a radioactive isotope, a therapeutic agent, or a combination thereof. In one embodiment, the cytotoxic agent comprises a calicheamicin, ozogamicin, monomethyl auristatin E, emtansine, a derivative or a combination thereof.

The imaging agent may be any compound useful for imaging purpose. In one embodiment, the imaging agent may be radionuclide, a florescent agent, a quantum dots, or a combination thereof.

The application further provides pharmaceutical composition. In one embodiment, the pharmaceutical composition comprises a pharmaceutically acceptable carrier and the tetra-specific antibody monomer disclosed herein. In one embodiment, the pharmaceutical composition comprises a pharmaceutically acceptable carrier and the multi-specific antibody disclosed herein. In one embodiment, the pharmaceutical composition comprises a pharmaceutically acceptable carrier and the antigen-binding fragment disclosed herein. In one embodiment, the pharmaceutical composition comprises a pharmaceutically acceptable carrier and the immuno-conjugate disclosed herein.

In one embodiment, the pharmaceutical composition further includes a therapeutic agent. Example therapeutic agents include without limitation a radioisotope, radionuclide, a toxin, a chemotherapeutic agent or a combination thereof. In one embodiment, the therapeutic agent comprises an antibody, an enzyme, or a combination thereof. In one embodiment, the therapeutic agent comprises an anti-estrogen agent, a receptor tyrosine kinase inhibitor, a kinase inhibitor, a cell cycle inhibitor, a DNA, RNA or protein synthesis inhibitor, a RAS inhibitor, or a combination thereof. In one embodiment, the therapeutic agent comprises a check point inhibitor. In one embodiment, the therapeutic agent comprises an inhibitor of PD1, PDL1, CTLA4, 4-1BB, OX40, GITR, ICOS, LIGHT, TIM3, LAG3, TIGIT, CD40, CD27, HVEM, BTLA, VISTA, B7H4, CSF1R, NKG2D, CD73, a derivative or a combination thereof.

In a further aspect, the application provides methods for making the tetra-specific antibody monomers, the multi-specific antibodies, the antigen-binding fragments thereof, and immuno-conjugates thereof. In one embodiment, the method includes the steps of culturing the host cell containing the nucleic acid sequences disclosed herein such that the DNA sequence encoding the antibody is expressed and purifying the antibody. In one embodiment, the antibody is a tetra-specific antibody.

In a further aspect, the application provides methods of using the tetra-specific antibody monomers, the multi-specific antibodies, the antigen-binding fragments thereof, and immuno-conjugates thereof for cancer treatment. In one embodiment, the method includes the step of administering tetra-specific antibody monomers, the multi-specific antibodies, the antigen-binding fragments thereof, and immuno-conjugates thereof, or pharmaceutical composition thereof to a subject in need of such treatment. In one embodiment, the method includes the step of administering to the subject an effective amount of the tetra-specific antibody.

In one embodiment, the method includes directly injecting into the tumour site an effective amount of multi-specific monomers, multi-specific antibodies, the immuno-conjugates, the antigen-binding fragment thereof.

Varieties of cancer may be prevented or treated. In one embodiment, the cancer may have cells expressing ROR1, CEA, HER2, EGFR, EGFR VIII, LMP1, LMP2A, Mesothelin, PSMA, EpCAM, glypimay-3, gpA33, GD2, TROP2, NKG2D, BCMA, CD19, CD20, CD33, CD123, CD22, or CD30. Example cancers include without limitation breast cancer, colorectal cancer, anal cancer, pancreatic cancer, gallbladder cancer, bile duct cancer, head and neck cancer, nasopharyngeal cancer, skin cancer, melanoma, ovarian cancer, prostate cancer, urethral cancer, lung cancer, non-small lung cell cancer, small cell lung cancer, brain tumor, glioma, neuroblastoma, oesophageal cancer, gastric cancer, liver cancer, kidney cancer, bladder cancer, cervical cancer, endometrial cancer, thyroid cancer, eye cancer, sarcoma, bone cancer, leukemia, myeloma or lymphoma.

In one embodiment, the method may further include co-administering an effective amount of a therapeutic agent. In one embodiment, the therapeutic agent may include an antibody, a chemotherapy agent, an enzyme, or a combination thereof. In one embodiment, the therapeutic agent may include an anti-estrogen agent, a receptor tyrosine kinase inhibitor, a kinase inhibitor, a cell cycle inhibitor, a DNA, RNA or protein synthesis inhibitor, a RAS inhibitor, or a combination thereof. In one embodiment, the therapeutic agent may include a check point inhibitor. In one embodiment, the therapeutic agent may include an inhibitor of PD1, PD-L1, CTLA4, 4-1BB, OX40, GITR, ICOS, LIGHT, TIM3, LAG3, TIGIT, CD40, CD27, HVEM, BTLA, VISTA, B7H4, CSF1R, NKG2D, CD73, a derivative or a combination thereof.

In one embodiment, the therapeutic agent may comprises capecitabine, cisplatin, Cyclophosphamide, methotrexate, 5-fluorouracil, Doxorubicin, cyclophosphamide, Mustine, vincristine, procarbazine, prednisolone, bleomycin, vinblastine, dacarbazine, etoposide, Epirubicin, pemetrexed, folinic acid, gemicitabine, oxaliplatin, irinotecan, topotecan, camptothecin, docetaxel, paclitaxel, fulvestrant, tamoxifen, letrozole, exemestane, anastrozole, aminoglutethimide, testolactone, vorozole, formestane, fadrozole, erlotinib, lafatinib, dasatinib, gefitinib, osimertinib, vandertanib, afatinib, imatinib, pazopinib, lapatinib, sunitinib, nilotinib, sorafenib, nab-palitaxel, Everolimus, temsirolimus, Dabrafenib, vemurafenib, trametinib, vintafolide, apatinib, crizotinib, periforsine, olaparib, Bortezomib, tofacitinib, trastuzumab, a derivative or a combination thereof.

The subject may be a human. In one embodiment, the subject may be suffering from cancer. The application further provides solutions comprising an effective concentration of the multi-specific antibodies, monomers, or immuno-conjugates disclosed herein. In one embodiment, the solution is blood plasma in a subject.

The objectives and advantages of the disclosure may become apparent from the following detailed description of example embodiments thereof in connection with the accompanying drawings. Still other embodiments may become readily apparent to those skilled in the art from the following detailed description, wherein are described embodiments by way of illustrating the best mode contemplated. As may be realized, other and different embodiments are possible and the embodiments' several details are capable of modifications in various obvious respects, all without departing from their spirit and the scope. Accordingly, the drawings and detailed description are to be regarded as illustrative in nature and not as restrictive.

### BRIEF DESCRIPTION OF FIGURES

The foregoing and other features of this disclosure may become more fully apparent from the following description and appended claims, taken in conjunction with the accompanying drawings. Understanding that these drawings depict only several embodiments arranged in accordance with the disclosure and are, therefore, not to be considered limiting of its scope, the disclosure may be described with additional specificity and detail through use of the accompanying drawings, in which:

FIG. 1 is a diagram of a tetra-specific antibody with Domains 1-4 as antigen binding domains. CD3×EGFRvIII×PD-L1×4-1BB tetra-specific antibody is shown as an example in accordance to one embodiment.

FIG. 2 depicts experiment results showing Redirected PBMC (peripheral blood mononuclear cells) cytotoxicity against astrocytoma cell line U87 that was transfected with EGFRvIII. Tumor lysis activity of tetra-specific antibodies used is listed in TABLE 1.

FIG. 3 depicts experiment results showing Redirected PBMC (peripheral blood mononuclear cells) cytotoxicity against acute lymphoblastic leukemia cell line Kasumi-2.

FIG. 4 depicts experiment results showing Redirected PBMC (peripheral blood mononuclear cells) cytotoxicity against astrocytoma cell line U87 that was transfected with EGFRvIII. Functional activities of different 4-1BB domains and functional impact of PD-L1 and 4-1BB domains are shown.

FIG. 5 is a depiction of experiment results showing FACS analysis of tetra-specific antibodies binding to human ROR1 transfected CHO cells according to some embodiments.

FIG. 6 is a depiction of experiment results showing FACS analysis of tetra-specific antibodies binding to human 4-1BB transfected CHO cells according to some embodiments.

FIG. 7 is a depiction of experiment results showing FACS analysis of tetra-specific antibodies binding to human PD-L1 transfected CHO cells according to some embodiments.

FIG. 8 is a depiction of experiment results showing redirected T cell cytotoxicity (RTCC) assay, meditated by tetra-specific antibodies with the binding domain 323H7 which is specific for the Ig domain of ROR1, with peripheral blood mononuclear cells as effectors and the B-ALL cell line Kasumi2 as targets according to some embodiments.

FIG. 8 is a depiction of experiment results showing redirected T cell cytotoxicity (RTCC) assay, meditated by tetra-specific antibodies with the binding domain 323H7 which is specific for the Ig domain of ROR1, with peripheral blood mononuclear cells as effectors and the B-ALL cell line Kasumi2 as targets according to some embodiments.

FIG. 9 is a depiction of experiment results showing redirected T cell cytotoxicity (RTCC) assay, meditated by tetra-specific antibodies with the binding domain 323H7 which is specific for the Ig domain of ROR1, with CD8+, CD45RO+ memory T cells as effectors and the B-ALL cell line Kasumi2 as targets according to some embodiments.

FIG. 10 is a depiction of experiment results showing redirected T cell cytotoxicity (RTCC) assay, meditated by tetra-specific antibodies with the binding domain 323H7 which is specific for the Ig domain of ROR1, with CD8+, CD45RA+ naive T cells as effectors and the B-ALL cell line Kasumi2 as targets according to some embodiments.

FIG. 11 is a depiction of experiment results showing redirected T cell cytotoxicity (RTCC) assay, meditated by tetra-specific antibodies with the binding domain 338H4 which is specific for the Frizzled domain of ROR1, with peripheral blood mononuclear cells as effectors and the B-ALL cell line Kasumi2 as targets according to some embodiments.

FIG. 12 is a depiction of experiment results showing redirected T cell cytotoxicity (RTCC) assay, meditated by tetra-specific antibodies with the binding domain 338H4 which is specific for the Frizzled domain of ROR1, with CD8+, CD45RO+ memory T cells as effectors and the B-ALL cell line Kasumi2 as targets according to some embodiments.

FIG. 13 is a depiction of experiment results showing redirected T cell cytotoxicity (RTCC) assay, meditated by tetra-specific antibodies with the binding domain 338H4 which is specific for the Frizzled domain of ROR1, with CD8+, CD45RA+ naive T cells as effectors and the B-ALL cell line Kasumi2 as targets according to some embodiments.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It may be readily understood that the aspects of the present disclosure, as generally described herein, and illustrated in the FIGUREs, may be arranged, substituted, combined, separated, and designed in a wide variety of different configurations, all of which are explicitly contemplated herein.

The disclosure provides, among others, isolated antibodies, methods of making such antibodies, tetra-specific or multi-specific molecules, antibody-drug conjugates and/or immuno-conjugates composed from such antibodies or antigen binding fragments, pharmaceutical compositions containing the antibodies, tetra-specific or multi-specific molecules, antibody-drug conjugates and/or immuno-conjugates, method of making thereof, and method of using the disclosed molecules or composition for treatment of cancer.

The term “antibody” is used in the broadest sense and specifically covers single monoclonal antibodies (including agonist and antagonist antibodies), antibody compositions with polyepitopic specificity, as well as antibody fragments (e.g., Fab, F(ab′)2, and Fv), so long as they exhibit the desired biological activity. In some embodiments, the antibody may be monoclonal, polyclonal, chimeric, single chain, tetra-specific or bi-effective, simianized, human and humanized antibodies as well as active fragments thereof. Examples of active fragments of molecules that bind to known antigens include Fab, F(ab′)2, scFv and Fv fragments, including the products of an Fab immunoglobulin expression library and epitope-binding fragments of any of the antibodies and fragments mentioned above. In some embodiments, antibody may include immunoglobulin molecules and immunologically active portions of immunoglobulin molecules, i.e. molecules that contain a binding site that immunospecifically bind an antigen. The immunoglobulin may be of any type (lgG, IgM, IgD, IgE, IgA and IgY) or class (IgG1, IgG2, IgG3, IgG4, IgA1 and IgA2) or subclasses of immunoglobulin molecule. In one embodiment, the antibody may be whole antibodies and any antigen-binding fragment derived from the whole antibodies. A typical antibody refers to hetero-tetrameric protein comprising typically of two heavy (H) chains and two light (L) chains. Each heavy chain is comprised of a heavy chain variable domain (abbreviated as VH) and a heavy chain constant domain. Each light chain is comprised of a light chain variable domain (abbreviated as VL) and a light chain constant domain. The VH and VL regions may be further subdivided into domains of hypervariable complementarity determining regions (CDR), and more conserved regions called framework regions (FR). Each variable domain (either VH or VL) is typically composed of three CDRs and four FRs, arranged in the following order: FR1, CDR1, FR2, CDR2, FR3, CDR3, FR4 from amino-terminus to carboxy-terminus. Within the variable regions of the light and heavy chains there are binding regions that interacts with the antigen.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies may be advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler & Milstein, Nature, 256:495 (1975), or may be made by recombinant DNA methods (see, e.g., U.S. Pat. No. 4,816,567).

The monoclonal antibodies may include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 [1984]).

Monoclonal antibodies may be produced using various methods including mouse hybridoma or phage display (see Siegel. Transfus. Clin. Biol. 9:15-22 (2002) for a review) or from molecular cloning of antibodies directly from primary B cells (see Tiller. New Biotechnol. 28:453-7 (2011)). In the present disclosure antibodies were created by the immunization of rabbits with both human PD-L1 protein and cells transiently expressing human PD-L1 on the cell surface. Rabbits are known to create antibodies of high affinity, diversity and specificity (Weber et al. Exp. Mol. Med. 49:e305). B cells from immunized animals were cultured in vitro and screened for the production of anti-PD-L1 antibodies. The antibody variable genes were isolated using recombinant DNA techniques and the resulting antibodies were expressed recombinantly and further screened for desired features such as ability to inhibit the binding of PD-L1 to PD-1, the ability to bind to non-human primate PD-L1 and the ability to enhance human T-cell activation. This general method of antibody discovery is similar to that described in Seeber et al. PLOS One. 9:e86184 (2014).

The term “antigen- or epitope-binding portion or fragment” refers to fragments of an antibody that are capable of binding to an antigen (PD-L1 in this case). These fragments may be capable of the antigen-binding function and additional functions of the intact antibody. Examples of binding fragments include, but are not limited to a single-chain Fv fragment (scFv) consisting of the VL and VH domains of a single arm of an antibody connected in a single polypeptide chain by a synthetic linker or a Fab fragment which is a monovalent fragment consisting of the VL, constant light (CL), VH and constant heavy 1 (CH1) domains. Antibody fragments may be even smaller sub-fragments and may consist of domains as small as a single CDR domain, in particular the CDR3 regions from either the VL and/or VH domains (for example see Beiboer et al., J. Mol. Biol. 296:833-49 (2000)). Antibody fragments are produced using conventional methods known to those skilled in the art. The antibody fragments are may be screened for utility using the same techniques employed with intact antibodies.

The “antigen- or epitope-binding fragments” may be derived from an antibody of the present disclosure by a number of art-known techniques. For example, purified monoclonal antibodies may be cleaved with an enzyme, such as pepsin, and subjected to HPLC gel filtration. The appropriate fraction containing Fab fragments may then be collected and concentrated by membrane filtration and the like. For further description of general techniques for the isolation of active fragments of antibodies, see for example, Khaw, B. A. et al. J. Nucl. Med. 23:1011-1019 (1982); Rousseaux et al. Methods Enzymology, 121:663-69, Academic Press, 1986.

Papain digestion of antibodies produces two identical antigen binding fragments, called “Fab” fragments, each with a single antigen binding site, and a residual “Fc” fragment, whose name reflects its ability to crystallize readily. Pepsin treatment yields an F(ab′)2 fragment that has two antigen combining sites and is still capable of cross-linking antigen.

The Fab fragment may contain the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other, chemical couplings of antibody fragments are also known.

“Fv” is the minimum antibody fragment which contains a complete antigen recognition and binding site. This region consists of a dimer of one heavy and one light chain variable domain in tight, non-covalent association. It is in this configuration that the three CDRs of each variable domain interact to define an antigen binding site on the surface of the VH-VL dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The “light chains” of antibodies (immunoglobulins) from any vertebrate species may be assigned to one of two clearly distinct types, called kappa and lambda (A), based on the amino acid sequences of their constant domains.

Depending on the amino acid sequence of the constant domain of their heavy chains, immunoglobulins may be assigned to different classes. There are five major classes of immunoglobulins: IgA, IgD, IgE, IgG and IgM, and several of these may be further divided into subclasses (isotypes), e.g., lgG-1, IgG-2, IgG-3, and IgG-4; IgA-1 and IgA-2. The heavy chain constant domains that correspond to the different classes of immunoglobulins are called a, delta, epsilon, y, and u, respectively. The subunit structures and three-dimensional configurations of different classes of immunoglobulins are well known.

A “humanized antibody” refers to a type of engineered antibody having its CDRs derived from a non-human donor immunoglobulin, the remaining immunoglobulin-derived parts of the molecule being derived from one (or more) human immunoglobulin(s). In some embodiments, framework support residues may be altered to preserve binding affinity. Methods to obtain “humanized antibodies” are well known to those skilled in the art. (see, e.g., Queen et al., Proc. Natl Acad Sci USA, 86:10029-10032 (1989), Hodgson et al., Bio/Technology, 9:421 (1991)).

The terms “polypeptide”, “peptide”, and “protein”, as used herein, are interchangeable and are defined to mean a biomolecule composed of amino acids linked by a peptide bond.

The terms “a”, “an” and “the” as used herein are defined to mean “one or more” and include the plural unless the context is inappropriate.

By “isolated” is meant a biological molecule free from at least some of the components with which it naturally occurs. “Isolated,” when used to describe the various polypeptides disclosed herein, means a polypeptide that has been identified and separated and/or recovered from a cell or cell culture from which it was expressed. Ordinarily, an isolated polypeptide may be prepared by at least one purification step. An “isolated antibody,” refers to an antibody which is substantially free of other antibodies having different antigenic specificities.

“Recombinant” means the antibodies are generated using recombinant nucleic acid techniques in exogeneous host cells.

The term “antigen” refers to an entity or fragment thereof which may induce an immune response in an organism, particularly an animal, more particularly a mammal including a human. The term includes immunogens and regions thereof responsible for antigenicity or antigenic determinants.

Also as used herein, the term “immunogenic” refers to substances which elicit or enhance the production of antibodies, T-cells or other reactive immune cells directed against an immunogenic agent and contribute to an immune response in humans or animals. An immune response occurs when an individual produces sufficient antibodies, T-cells and other reactive immune cells against administered immunogenic compositions of the present disclosure to moderate or alleviate the disorder to be treated.

“Specific binding” or “specifically binds to” or is “specific for” a particular antigen or an epitope means binding that is measurably different from a non-specific interaction. Specific binding may be measured, for example, by determining binding of a molecule compared to binding of a control molecule, which generally is a molecule of similar structure that does not have binding activity. For example, specific binding may be determined by competition with a control molecule that is similar to the target.

Specific binding for a particular antigen or an epitope may be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen may have a KD that is 20-, 50-, 100-, 500-, 1000-, 5,000-, 10,000- or more times greater for a control molecule relative to the antigen or epitope.

Also, specific binding for a particular antigen or an epitope may be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction.

“Homology” between two sequences is determined by sequence identity. If two sequences which are to be compared with each other differ in length, sequence identity preferably relates to the percentage of the nucleotide residues of the shorter sequence which are identical with the nucleotide residues of the longer sequence. Sequence identity may be determined conventionally with the use of computer programs. The deviations appearing in the comparison between a given sequence and the above-described sequences of the disclosure may be caused for instance by addition, deletion, substitution, insertion or recombination.

In one aspect, the application provides tetra-specific antibody monomers, antigen-binding fragments, and multi-specific antibodies. In one embodiment, the application provides tetra-specific antibodies.

In one embodiment, the disclosure provides tetra-specific antibodies with a binding specificity against four different antigen targets. In one embodiment, the antigen targets are tumor specific antigens, T cell receptor CD3 component, or immune checkpoint molecules. The tetra-specific antibodies may directly engage body's endogenous T cells to kill tumor cells independent of tumor antigen presentation by MHC to the antigen specific T cell receptors. In some embodiments, the immune checkpoint modulating component of the tetra-specific antibodies may overcome the immunosuppressive tumor microenvironment to fully activate the exhausted T cells within the tumor microenvironment.

The tetra-specific antibodies have unique properties of directly engaging T cells at the same time modulating immune checkpoint or inhibiting Treg or other inhibitory immune cells or targeting tumor with component against tumor antigens. It may show benefit to the patients where BiTE or CAR-T treatment isn't appropriate. In one embodiment, the tetra-specific antibodies could demonstrate clinical benefit in solid tumor where BiTE-like technology or CAR-T treatment yet to show clinical benefit due to the limitations imposed by the inhibitory tumor microenvironment.

In one embodiment, the application provides an engineered antibody with 4 different binding domains or a “tetra-specific antibody”. One binding domain is specific for CD3 on T cells, a second binding domain is specific for a tumor associated antigen including but not limited to ROR1, CEA, HER2, EGFR, EGFRvIII, LMP1, LMP2A, Mesothelin, PSMA, EpCAM, glypimay-3, gpA33, GD2, TROP2, BCMA, CD19, CD20, CD33, CD123, CD22, CD30, and a third and fourth binding domains are specific for two distinct immune checkpoint modulators such as PD-L1, PD-1, OX40, 4-1BB, GITR, TIGIT, TIM-3, LAG-3, CTLA4, CD40, VISTA, ICOS, BTLA, Light, HVEM, CD73, CD39, etc.

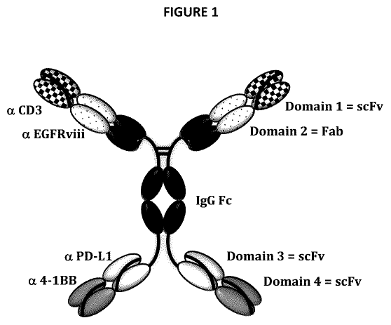

An example tetra-specific molecules disclosed herein (FIG. 1) target either human ROR1 (SEQIDs 33-48), human CD19 (SEQIDs 53-56) or EGFR vIII (SEQIDs 49-52) as tumor associated antigens. Each of these targeted tetra-specific proteins also carries an anti-human PD-L1 (SEQIDs 9-16), an anti-human 4-1BB (SEQIDs 21-32) and an anti-human CD3 binding domain (SEQIDs 1-8). These binding domains were converted to scFv, VLVH, for placement at the N-terminal Domain 1 (D1) or scFv, VHVL, for placement at the C-terminal Domains 3 (D3) and 4 (D4) of the peptide (FIG. 1).

In some embodiments, scFv molecules described herein contain a 20 amino acid flexible gly-gly-gly-gly-ser (G4S) X4 linker that operably links the VH and VL, regardless of the V-region orientation (LH or HL). The remaining position in the tetra-specific protein, Domain 2 (D2), consists of an IgG1 heavy chain, VH-CH1-Hinge-CH2-CH3, and its corresponding light chain, VL-CL, which may be either a kappa or lambda chain. D1 and D2 are genetically linked through a 10 amino acid (G4S)×2 linker, as are D2, D3 and D4 resulting in a contiguous ˜150 kDa heavy chain monomer peptide. When co-transfected with the appropriate light chain, the final symmetric tetra-specific peptide may be purified through the IgG1 Fc (Protein A/Protein G) and assayed to assess functional activity. Heavy and light chain gene “cassettes” were previously constructed such that V-regions could be cloned using either restriction enzyme sites (HindIII/NheI for the heavy chain and HindIII/BsiWI for the light chain) or “restriction-free cloning” such as GIBSON ASSEMBLY™ (SGI-DNA, La Jolla, CA), Infusion (Takara Bio USA) or NEBUILDER® (NEB, Ipswich, MA), the latter of which was used here.

Tetra-specific proteins are produced through a process that involves design of the intact molecule, synthesis and cloning of the nucleotide sequences for each domain, expression in mammalian cells and purification of the final product. Nucleotide sequences were assembled using the GENEIOUS® 10.2.3 software package (Biomatters, Auckland, NZ) and broken up into their component domains for gene synthesis (GENEWIZ®, South Plainsfield, NJ).

In this example, SI-35E18 (SEQID 65 and 67) was split into its component domains where the anti-4-1BB scFv, VLVH, occupies D1, anti-human PD-L1 clone PL230C6 occupies D2 (Fab position), anti-human ROR1 Ig domain-specific clone 323H7 VHVL scFv occupies D3, and anti-human CD3 scFv, VHVL, occupies the C-terminal D4. Using NEBUILDER® web-based tools, 5′ and 3′ nucleotides were appended to each of the domains depending on their position in the larger protein so that each domain overlaps its flanking domains by 20-30 nucleotides which direct site-specific recombination, thus genetically fusing each domain in a single gene assembly step. Due to the high number of homologous regions in the tetra-specific nucleotide sequence, the N-terminal domains 1 and 2 are assembled separately from the C-terminal D3 and D4. The N- and C-terminal fragments were then assembled together in a second NEBUILDER® reaction.

A small aliquot was transformed into E. coli DH10b (Invitrogen, Carlsbad, CA) and plated on TB+carbenicillin 100 ug/ml plates (Teknova, Hollister, CA) and incubated at 37 C overnight. Resultant colonies were selected and 2 ml overnight cultures inoculated in TB+carbenicillin. DNA was prepared (Thermo-Fisher, Carlsbad, CA) from overnight cultures and subsequently sequenced (GENEWIZ®, South Plainsfield, NJ) using sequencing primers (Sigma, St. Louis, MO) flanking each domain. In some embodiments, DNA sequences were assembled and analyzed in GENEIOUS® software.

In another aspect, the application provides pharmaceutical compositions including the multi-specific antibody monomers, the multi-specific antibodies, the antigen-binding fragments, and the immuno-conjugates thereof, and methods of using the disclosed antibodies or pharmaceutical compositions for treatment of cancer.

The advantages of using the disclosed tetra-specific antibody monomers, multi-specific antibodies or compositions for treatment purpose over any existing therapies include, among others: 1) Inclusion of an IgG Fc domain may confer the characteristic of a longer half-life in serum compared to a bi-specific BiTe molecule; 2) Inclusion of two binding domains that are specific for immune checkpoint modulators, that may inhibit the suppressive pathways and engage the co-stimulatory pathways at the same time; and 3) Cross-link CD3 on T cells with tumor associated antigens thus “re-directing” T cells to kill the tumor without the need to remove T cells from the patient and genetically modify them to be specific for the tumor cell before re-introducing them back into the patient as done for chimeric antigen receptor T cells (CAR-T).

Formulation of the pharmaceutical composition may be accomplished according to standard methodology know to those of ordinary skill in the art.

In one embodiment, the antibodies and monomers according to the disclosure may be prepared in a physiologically acceptable formulation and may comprise a pharmaceutically acceptable carrier, diluent and/or excipient using known techniques. For example, the antibody according to the disclosure and as described herein including any functionally equivalent antibody or functional parts thereof, in particular, the monoclonal antibody including any functionally equivalent antibody or functional parts thereof is combined with a pharmaceutically acceptable carrier, diluent and/or excipient to form a therapeutic composition. Formulation of the pharmaceutical composition according to the disclosure may be accomplished according to standard methodology know to those of ordinary skill in the art.

With respect to the formulation of suitable compositions for administration to a subject such as a human patient in need of treatment, the antibodies disclosed herein may be mixed or combined with pharmaceutically acceptable carriers known in the art dependent upon the chosen route of administration. There are no particular limitations to the modes of application of the antibodies disclosed herein, and the choice of suitable administration routes and suitable compositions are known in the art without undue experimentation.

Suitable pharmaceutical carriers, diluents and/or excipients are well known in the art and include, for example, phosphate buffered saline solutions, water, emulsions such as oil/water emulsions.

“Pharmaceutically acceptable” refers to those compounds, materials, compositions, and dosage forms which are, within the scope of sound medical judgment, suitable for use contact with the tissues of human beings or animals without excessive toxicity, irritation, or other problem or complication, commensurate with a reasonable benefit/risk ratio.

In one embodiment, the pharmaceutical composition may include proteinaceous carriers such as, for example, serum albumin or immunoglobulin, particularly of human origin. Further biologically active agents may be present in the pharmaceutical composition of the disclosure dependent on the intended use. In one embodiment, the proteinaceous pharmaceutically active matter may be present in amounts between 1 ng and 10 mg per dose. Generally, the regime of administration should be in the range of between 0.1 μg and 10 mg of the antibody according to the disclosure, particularly in a range 1.0 μg to 1.0 mg, and more particularly in a range of between 1.0 μg and 100 μg, with all individual numbers falling within these ranges also being part of the disclosure. If the administration occurs through continuous infusion a more proper dosage may be in the range of between 0.01 μg and 10 mg units per kilogram of body weight per hour with all individual numbers falling within these ranges also being part of the disclosure.

The compositions of the present disclosure may be administered to a subject in the form of a solid, liquid or aerosol at a suitable, pharmaceutically effective dose. Examples of solid compositions include pills, creams, and implantable dosage units. Pills may be administered orally. Therapeutic creams may be administered topically. Implantable dosage units may be administered locally, for example, at a tumor site, or may be implanted for systematic release of the therapeutic composition, for example, subcutaneously. Examples of liquid compositions include formulations adapted for injection intramuscularly, subcutaneously, intravenously, intra-arterially, and formulations for topical and intraocular administration. Examples of aerosol formulations include inhaler formulations for administration to the lungs.

It is well known to those of ordinary skill in the art that the dosage of the composition may depend on various factors such as, for example, the condition of being treated, the particular composition used, and other clinical factors such as weight, size, sex and general health condition of the patient, body surface area, the particular compound or composition to be administered, other drugs being administered concurrently, and the route of administration.

The term “therapeutically effective amount” or “effective amount” refers to the amount of antibody which, when administered to a human or animal, elicits a response which is sufficient to result in a therapeutic effect in said human or animal, e.g., to ameliorate disease in a subject. The effective amount is readily determined by one of ordinary skill in the art following routine procedures. Where the disease is a cancer, the effective amount of the drug may inhibit (for example, slow to some extent, inhibit or stop) one or more of the following example characteristics including, without limitation, cancer cell growth, cancer cell proliferation, cancer cell motility, cancer cell infiltration into peripheral organs, tumor metastasis, and tumor growth. Wherein the disease is a mayer, the effective amount of the drug may alternatively do one or more of the following when administered to a subject: slow or stop tumor growth, reduce tumor size (for example, volume or mass), relieve to some extent one or more of the symptoms associated with the cancer, extend progression free survival, result in an objective response (including, for example, a partial response or a complete response), and increase overall survival time. To the extent the drug may prevent growth and/or kill existing cancer cells, it is cytostatic and/or cytotoxic.

A person skilled in the art have the ability to determine the effective amount or concentration of the antibodies disclosed therein to effective treat a condition such as a cancer. Other parameters such as the proportions of the various components in the pharmaceutical composition, administration does and frequency may be obtained by a person skilled in the art without undue experimentation. For example, a suitable solution for injection may contain, without limitation, from about 1 to about 20, from about 1 to about 10 mg antibodies per ml. The example dose may be, without limitation, from about 0.1 to about 20, from about 1 to about 5 mg/Kg body weight. The example administration frequency could be, without limitation, once per day or three times per week.

The compositions may be administered by standard routes of administration. In general, the composition may be administered by topical, oral, rectal, nasal, intradermal, intraperitoneal, or parenteral (for example, intravenous, subcutaneous, or intramuscular) routes. In some embodiments, the composition may be incorporated into sustained release matrices such as biodegradable polymers, the polymers being implanted in the vicinity of where delivery is desired, for example, at the site of a tumor. The method includes administration of a single dose, administration of repeated doses at predetermined time intervals, and sustained administration for a predetermined period of time.

Although many forms of administration are possible, an example administration form would be a solution for injection, in particular for intravenous or intra-arterial injection. Usually, a suitable pharmaceutical composition for injection may include pharmaceutically suitable carriers or excipients such as, without limitation, a buffer, a surfactant, or a stabilizer agent. Example buffers may include, without limitation, acetate, phosphate or citrate buffer. Example surfactants may include, without limitation, polysorbate. Example stabilizer may include, without limitation, human albumin.

In one embodiment, the administration may be parenterally, e.g. intravenously. Preparations for parenteral administration include sterile aqueous or non-aqueous solutions, suspensions and emulsions. Non-aqueous solvents include without being limited to it, propylene glycol, polyethylene glycol, vegetable oil such as olive oil, and injectable organic esters such as ethyl oleate. Aqueous solvents may be chosen from the group consisting of water, alcohol/aqueous solutions, emulsions or suspensions including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose) and others. Preservatives may also be present such as, for example, antimicrobials, anti-oxidants, chelating agents, inert gases, etc.

The antibody monomers, antibodies, antigen-binding fragments and immuno-conjugates thereof may be used in combination with a therapeutic agent or a composition comprising a therapeutic agent for treatment purpose.

In some embodiments, the multi-specific antibody molecule is used in combination with one or more additional therapeutic agents at an effective amount. The additional therapeutic agent includes an antibody, a chemotherapy agent, an enzyme, or a combination thereof. In some embodiment, the additional therapeutic agent may be an anti-estrogen agent, a receptor tyrosine kinase inhibitor, a kinase inhibitor, a cell cycle inhibitor, a DNA, RNA or protein synthesis inhibitor, a RAS inhibitor, or a combination thereof. In some embodiments, the additional therapeutic agent may be a check point inhibitor. In some embodiments, therapeutic agent comprises inhibitors of PD1, PDL1, CTLA4, 4-1BB, OX40, GITR, ICOS, LIGHT, TIM3, LAG3, TIGIT, CD40, CD27, HVEM, BTLA, VISTA, B7H4, CSF1R, NKG2D, CD73, a derivative or a combination thereof.

In one embodiment, the therapeutic agent may include capecitabine, cisplatin, trastuzumab, fulvestrant, tamoxifen, letrozole, exemestane, anastrozole, aminoglutethimide, testolactone, vorozole, formestane, fadrozole, letrozole, erlotinib, lafatinib, dasatinib, gefitinib, imatinib, pazopinib, lapatinib, sunitinib, nilotinib, sorafenib, nab-palitaxel, a derivative or a combination thereof. In one embodiment, the therapeutic agent may include capecitabine, cisplatin, Cyclophosphamide, methotrexate, 5-fluorouracil, Doxorubicin, cyclophosphamide, mustine, vincristine, procarbazine, prednisolone, bleomycin, vinblastine, dacarbazine, etoposide, Epirubicin, pemetrexed, folinic acid, gemicitabine, oxaliplatin, irinotemay, topotemay, camptothecin, docetaxel, paclitaxel, fulvestrant, tamoxifen, letrozole, exemestane, anastrozole, aminoglutethimide, testolactone, vorozole, formestane, fadrozole, letrozole, erlotinib, lafatinib, dasatinib, gefitinib, osimertinib, vandertanib, afatinib, imatinib, pazopinib, lapatinib, sunitinib, nilotinib, sorafenib, nab-palitaxel, Everolimus, temsirolimus, Dabrafenib, vemurafenib, trametinib, vintafolide, apatinib, crizotinib, periforsine, olaparib, Bortezomib, tofacitinib, a derivative or a combination thereof.

Cancers, including breast cancer, colorectal cancer, pancreatic cancer, head and neck cancer, melanoma, ovarian cancer, prostate cancer, non-small lung cell cancer, glioma, esophageal cancer, nasopharyngeal cancer, anal cancer, rectal cancer, gastric cancer, bladder cancer, cervical cancer, or brain cancer, may express cancer-associated genes. Inhibition of cancer-associated activity with specific monoclonal antibodies or antigen-binding fragment may have therapeutic effect on cancers. Furthermore, administering a therapeutically effective amount of composition comprising monoclonal antibodies or antigen-binding fragment specific for cancer-associated protein may cure, prevent, ameliorate, and delay the development or metastasis of cancers, through the effect of the cytotoxic agent.

The present disclosure may be understood more readily by reference to the following detailed description of specific embodiments and examples included herein. Although the present disclosure has been described with reference to specific details of certain embodiments thereof, it is not intended that such details should be regarded as limitations upon the scope of the disclosure.

### EXAMPLES

### Example 1: Binding of Tetra-Specific Antibodies to EGFRvIII Antigen

Binding of tetra-specific antibodies listed in TABLE 1 to EGFRvIII antigen expressed on the surface of U87 cell line was assessed using FACS method. The tetra-specific antibodies were incubated with U87 cell line and then detected with secondary anti-human antibodies directly conjugated to ALEXA FLUOR 647™ fluorochrome. Cellular binding of the tetra-specific antibodies was analyzed on a flow cytometer BD LSRFORTESSA™. All tested antibodies bound to the antigen with a KD in a single digit and sub-nanomolar range (TABLE 2). Observed differences in binding were within 3-fold range and might be driven by the position of the binding domain within the molecule as well as by interactions with adjacent domains.

TABLE 1 shows example tetra-specific antibodies with EGFRvIII tumor antigen binding domain. TABLE 2 shows the binding to EGFRvIII antigen expressed in U87 cell line. Binding of tetra-specific antibodies listed in TABLE 1 to EGFRvIII antigen was assessed by flow cytometry.

TABLE 1
Tetra-specific antibodies with EGFRvIII
tumor antigen binding domain.

| Antibody ID | Antibody domain structure |
|---|---|
| SI-39E29 | 806 × 284A10 × PL221 × 420H5 |
| SI-39E18 | 284A10 × 806 × PL221 × 420H5 |
| SI-39E13 | 420H5 × PL230 × 284A10 × 806 |
| SI-39E4 | PL230 × 806 × 284A10 × 420H5 |
| SI-39E10 | 420H5 × PL230 × 806 × 284A10 |
| SI-39E23 | PL230 × 806 × 420H5 × 284A10 |

TABLE 2
Binding to EGFRvIII antigen expressed in CHO cell line.

CHO-EGFRviii binding

| Antibody ID | Antibody domain structure | EC50 (nM) |
|---|---|---|
| SI-39E29 | 806 × 284A10 × PL221 × 420H5 | 0.489 |
| SI-39E18 | 284A10 × 806 × PL221 × 420H5 | 0.7549 |
| SI-39E13 | 420H5 × PL230 × 284A10 × 806 | 1.244 |
| SI-39E4 | PL230 × 806 × 284A10 × 420H5 | 1.115 |
| SI-39E10 | 420H5 × PL230 × 806 × 284A10 | 1.542 |
| SI-39E23 | PL230 × 806 × 420H5 × 284A10 | 0.9222 |

### Example 2: Binding of Tetra-Specific Antibodies to EGFRvIII, 4-1BB, PD-L1 and CD3 Protein Antigens

Binding affinities and kinetics of tetra-specific antibodies listed in TABLE 1 to their respective antigens was assessed via Surface Plasmon Resonance on FORTEBIO® OCTET® RED96 instrument. The antigens were immobilized on the sensor chip surface and the tested antibodies were flown over the immobilized antigens. All molecules showed high binding to the antigens (TABLE 3). SI-39E29, SI-39E18 and SI-39E23 showed lower binding to CD3 e/d antigen than other antibodies tested. TABLE 3 shows the binding of tetra-specific antibodies listed in TABLE 1 to EGFRvIII, 4-1BB, PD-L1 and CD3 antigens.

TABLE 3
Binding to EGFRvIII, 4-1BB, PD-L1 and CD3 antigens.
Table 3A.

| | | EGFRVIII binding | | | | 4-1BB binding | | | |
|---|---|---|---|---|---|---|---|---|---|
| Antibody ID | Antibody domain structure | Response | KD (M) | kon(1/Ms) | kdis(1/s) | Response | KD (M) | kon(1/Ms) | kdis(1/s) |
| SI-39E29 | 806 × 284A10 × PL221 × 420H5 | 1.4113 | <1.0E−12 | 1.38E+05 | <1.0E−07 | 1.9181 | 5.94E−10 | 2.32E+05 | 1.38E−04 |
| SI-39E18 | 284A10 × 806 × PL221 × 420H5 | 1.4606 | <1.0E−12 | 1.19E+05 | <1.0E−07 | 2.0165 | 3.93E−10 | 2.35E+05 | 9.24E−05 |
| SI-39E13 | 420H5 × PL230 × 284A10 × 806 | 1.2317 | <1.0E−12 | 9.90E+04 | <1.0E−07 | 2.4703 | 1.17E−10 | 2.27E+05 | 2.65E−05 |
| SI-39E4 | PL230 × 806 × 284A10 × 420H5 | 1.4058 | <1.0E−12 | 8.43E+04 | <1.0E−07 | 2.417 | 3.54E−10 | 1.67E+05 | 5.91E−05 |
| SI-39E10 | 420H5 × PL230 × 806 × 284A10 | 1.1414 | <1.0E−12 | 8.63E+04 | <1.0E−07 | 2.6348 | 3.56E−11 | 2.09E+05 | 7.43E−06 |
| SI-39E23 | PL230 × 806 × 420H5 × 284A10 | 1.3408 | <1.0E−12 | 1.41E+05 | <1.0E−07 | 2.0159 | 6.96E−10 | 2.03E+05 | 1.41E−04 |

Table 3B.

| | | PD-L1 binding | | | | CD3 e/d binding | | | |
|---|---|---|---|---|---|---|---|---|---|
| Antibody ID | Antibody domain structure | Response | KD (M) | kon(1/Ms) | kdis(1/s) | Response | KD (M) | kon(1/Ms) | kdis(1/s) |
| SI-39E29 | 806 × 284A10 × PL221 × 420H5 | 1.7228 | 2.83E−10 | 3.76E+05 | 1.06E−04 | 1.8493 | 4.75E−10 | 1.66E+05 | 7.86E−05 |
| SI-39E18 | 284A10 × 806 × PL221 × 420H5 | 1.8256 | 1.68E−10 | 3.79E+05 | 6.37E−05 | 1.7586 | 6.32E−10 | 1.74E+05 | 1.10E−04 |
| SI-39E13 | 420H5 × PL230 × 284A10 × 806 | 1.9223 | 8.01E−11 | 4.15E+05 | 3.32E−05 | 1.8019 | <1.0E−12 | 1.04E+05 | <1.0E−07 |
| SI-39E4 | PL230 × 806 × 284A10 × 420H5 | 2.1532 | 7.23E−11 | 3.33E+05 | 2.41E−05 | 1.965 | <1.0E−12 | 8.61E+04 | <1.0E−07 |
| SI-39E10 | 420H5 × PL230 × 806 × 284A10 | 1.9364 | 6.85E−11 | 3.81E+05 | 2.61E−05 | 2.2404 | <1.0E−12 | 1.10E+05 | <1.0E−07 |
| SI-39E23 | PL230 × 806 × 420H5 × 284A10 | 1.9024 | 4.02E−11 | 4.83E+05 | 1.94E−05 | 2.6545 | 2.07E−10 | 1.23E+05 | 2.54E−05 |

### Example 3: Redirected PBMC Cytotoxicity Against Astrocytoma Cell Line U87 that was Transfected with EGFRvIII

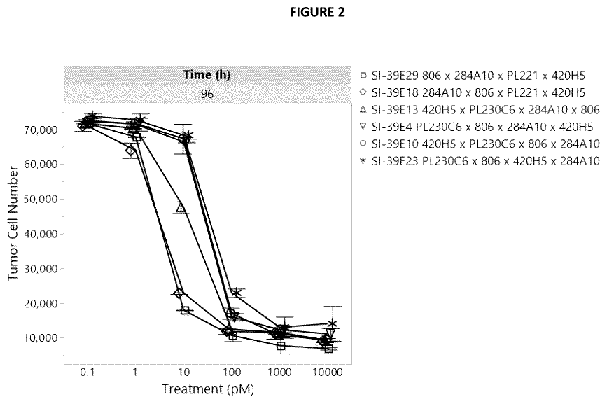

Tetra-specific antibodies listed in TABLE 1 were assessed for their ability to redirect PBMC to lyse U87 transfected with EGFRvIII tumor cell line (U87vIII). PBMC were isolated by ficoll gradient. U87vIII tumor cell line was stably expressing nucleus-localized Red Fluorescent Protein (RFP) delivered via lentiviral transduction (Sartorius). U87vIII tumor cells were co-cultured with PBMC. Lysis of tumor cells was assessed by counting RFP labeled tumor cell nuclei. Images were acquired on live cell imager INCUCYTE® (Sartorius). SI-39E18 and SI-39E13 tetra-specific antibodies showed the highest efficacy at 96 hours followed by SI-39E10. SI-39E4, SI-39E23 and SI-39E29 showed lower efficacy in this study than other antibodies listed in TABLE 1 (FIG. 2).

### Example 4: Redirected PBMC Cytotoxicity Against Acute Lymphoblastic Leukemia Cell Line Kasumi-2

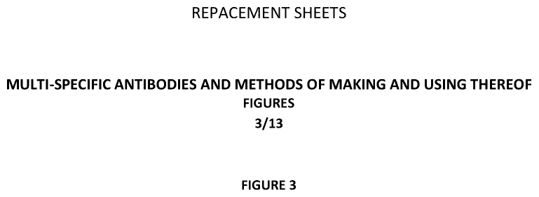

Tetra-specific antibodies listed in TABLE 4 were assessed for their ability to lyse leukemia cell line Kasumi-2. PBMC were isolated by ficoll gradient. Kasumi-2 tumor cells were co-cultured with PBMC. Tumor cell lysis was assessed on BD LSRFORTESSA® flow cytometer via counting the number of live tumor cells present after 96 hours of co-culture. Tetra-specific antibody SI-38E14 showed the most potent activity in this study followed by SI-38E38 (FIG. 3). TABLE 4 shows example tetra-specific antibodies with CD19 tumor antigen recognition domain.

TABLE 4
Tetraspecific antibodies with CD19
tumor antigen recognition domain.

| Antibody ID | Antibody domain structure |
|---|---|
| SI-38E14 | PL230 × 466F6 × 21D4 × 284A10 |
| SI-38E38 | PD224 × 466F6 × 21D4 × 284A10 |
| SI-38E5 | 466F6 × PL230 × 284A10 × 21D4 |
| SI-38E20 | 466F6 × 21D4 × 284A10 × PL221 |
| SI-38E35 | 21D4 × 284A10 × 466F6 × PL221 |

### Example 5: Redirected PBMC Cytotoxicity Against Astrocytoma Cell Line U87 that was Transfected with EGFRvIII, Functional Activity of Different 4-1BB Domains and Functional Impact of PD-L1 and 4-1BB Domains

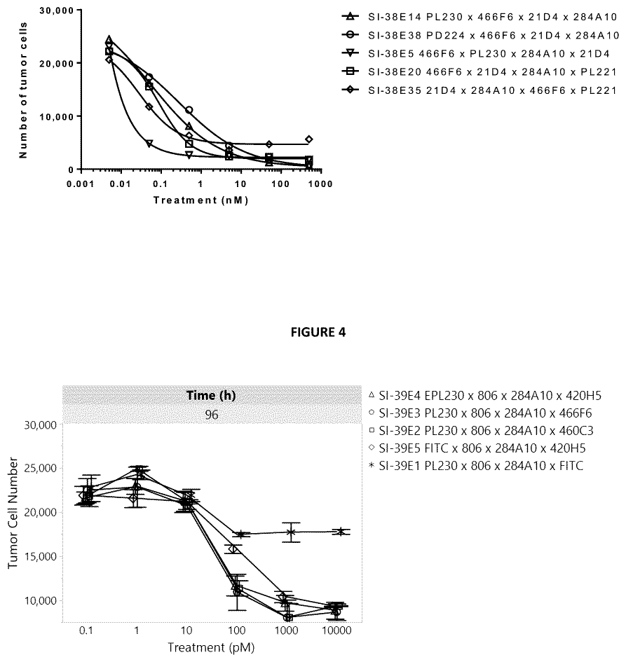

Tetra-specific antibodies listed in TABLE 5 were assessed for their ability to redirect PBMC to lyse U87 transfected with EGFRvIII tumor cell line (U87vIII). PBMC were isolated by ficoll gradient. U87vIII tumor cell line was stably expressing nucleus-localized Red Fluorescent Protein (RFP) delivered via lentiviral transduction (Sartorius). U87vIII tumor cells were co-cultured with PBMC. Lysis of tumor cells was assessed by counting RFP labeled tumor cell nuclei. Images were acquired on live cell imager INCUCYTE® (Sartorius). Activity of the antibodies was assessed after 96 hours of incubation. Antibodies with different 4-1BB domains—SI-39E4, SI-39E2 and SI-39E3 showed similar activity (FIG. 4). Antibodies with PD-L1 and 4-1BB domains replaced by silent (not functional) FITC domains, SI-39E1 and SI-39E5, showed reduction in lysis activity. This observation confirms functional contribution of 4-1BB and PD-L1 domains. TABLE 5 shows example tetra-specific antibodies with EGFRvIII tumor antigen binding domain. FITC control antibodies.

TABLE 5
Tetraspecific antibodies with EGFRvIII tumor antigen
binding domain. FITC control antibodies.

| Antibody ID | Antibody domain structure |
|---|---|
| SI-39E4 | PL230 × 806 × 284A10 × 420H5 |
| SI-39E3 | PL230 × 806 × 284A10 × 466F6 |
| SI-39E2 | PL230 × 806 × 284A10 × 460C3 |
| SI-39E5 | FITC × 806 × 284A10 × 420H5 |
| SI-39E1 | PL230 × 806 × 284A10 × FITC |

### Example 6: FACS Analysis of Tetra-Specific Specific Antibody Binding to Human ROR1 Transfected CHO Cells

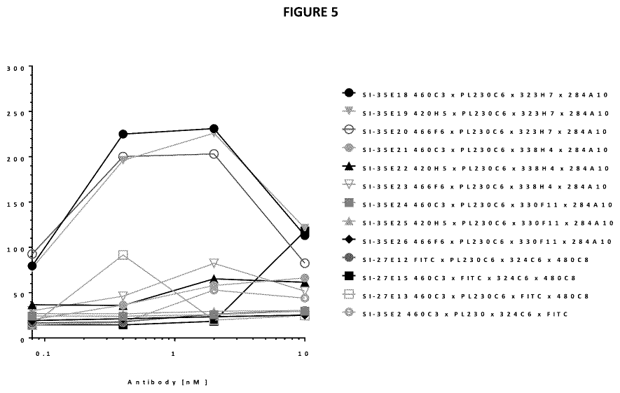

The tetra-specific-specific antibodies listed in TABLEs 1 and 2 were tested for binding to Chinese hamster ovary cells (CHO) cells stably expressing full length human ROR1. Antibodies were prepared at 2× final concentration and titrated 1:5 across 3 wells of a 96 well plate in 50 ul of PBS/2% FBS and then 5,000 ROR1-CHO cells in 50 ul PBS/2% FBS were added. This mixture was incubated for 30 minutes on ice, washed once with 200 ul PBS/2% FBS, and then the secondary antibody PE Goat anti-Human IgG Fc at 1:1000 dilution of stock was added, and this mixture was incubated for 30 minutes on ice. Cells were washed 2×200 ul PBS/2% FBS, resuspended in 50 ul PBS/2% FBS and analyzed on a BD LSRFORTESSA® and the binding profile is shown in FIG. 5. The tetra-specific antibodies SI-35E18, 19, and 20, with the 323H7 binding domain specific for the Ig domain of ROR1, showed higher binding than the tetra-specific antibodies SI-3521, 22, and 23, with the 338H4 binding domain specific for the frizzled domain of ROR1, and the tetra-specific antibodies SI-3524, 25, and 26, with the 330F11 binding domain specific for the kringle domain of ROR1, did not bind.

### Example 7: FACS Analysis of Tetra-Specific Specific Antibody Binding to Human 41BB Transfected CHO Cells

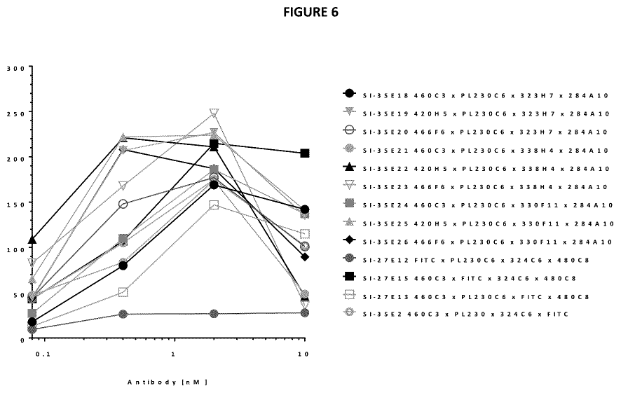

The tetra-specific-specific antibodies listed in TABLE 6 were tested for binding to Chinese hamster ovary cells (CHO) cells stably expressing full length human ROR1. Antibodies were prepared at 2× final concentration and titrated 1:5 across 3 wells of a 96 well plate in 50 ul of PBS/2% FBS and then 5,000 ROR1-CHO cells in 50 ul PBS/2% FBS were added. This mixture was incubated for 30 minutes on ice, washed once with 200 ul PBS/2% FBS, and then the secondary antibody PE Goat anti-Human IgG Fc at 1:1000 dilution of stock was added, and this mixture was incubated for 30 minutes on ice. Cells were washed 2×200 ul PBS/2% FBS, resuspended in 50 ul PBS/2% FBS and analyzed on a BD LSRFORTESSA® and the binding profile is shown in FIG. 6. All of the tetra-specific antibodies except for the control SI-27E12 contain a 41BB binding domain, 460C3, 420H5, or 466F6 and bound to 41BB expressing CHO cells with varying intensity. TABLE 6 shows the example tetra-specific antibody list.

TABLE 6
Tetraspecific antibody list.

| | | Domain 1 | | Domain 2 | | | Domain 3 | | Domain 4 | |
|---|---|---|---|---|---|---|---|---|---|---|
| | | | Humanized | | Humanized | | | Humanized | | Humanized |
| ID = SI-xx | | LH-scFv | Variant | Fab | Variant | Fc | HL-scFv | Variant | HL-scFv | Variant |
| 39 | E02 | PL230C6 | L2H3 | 806 | — | | 284A10 | H1L1 | 460C3 | H1L1 |
| 39 | E03 | PL230C6 | L2H3 | 806 | — | | 284A10 | H1L1 | 466F6 | H2L5 |
| 39 | E18 | 284A10 | L1H1 | 806 | — | n2 | PL221G5 | H1L1 | 420H5 | H3L3 |
| 39 | E13 | 420H5 | L3H3 | PL230C6 | H3L2 | n2 | 284A10 | H1L1 | 806 | |
| 39 | E10 | 420H5 | L3H3 | PL230C6 | H3L2 | n2 | 806 | — | 284A10 | H1L1 |
| 39 | E29 | 806 | — | 284A10 | H1L1 | n2 | PL221G5 | H1L1 | 420H5 | H3L3 |
| 39 | E04 | PL230C6 | L2H3 | 806 | — | n2 | 284A10 | H1L1 | 420H5 | H3L3 |
| 39 | E23 | PL230C6 | L2H3 | 806 | — | n2 | 420H5 | H3L3 | 284A10 | H1L1 |
| 38 | E14 | PL230C6 | L2H3 | 466F6 | H2L5 | n2 | 21D4 | — | 284A10 | H1L1 |
| 38 | E38 | PD224D1 | L2H2 | 466F6 | H2L5 | n2 | 21D4 | — | 284A10 | H1L1 |
| 38 | E05 | 466F6 | L5H2 | PL230C6 | H3L2 | n2 | 284A10 | H1L1 | 21D4 | — |
| 38 | E20 | 466F6 | L5H2 | 21D4 | — | n2 | 284A10 | H1L1 | PL221G5 | H1L1 |
| 39 | E05 | 4-4-20 | — | 806 | — | n2 | 284A10 | H1L1 | 420H5 | H3L3 |
| | | (FITC) | | | | | | | | |
| 39 | E01 | PL230C6 | L2H3 | 806 | — | n2 | 284A10 | H1L1 | 4-4-20 | — |
| | | | | | | | | | (FITC) | |
| 35 | E02 | 460C3 | L1H1 | PL230C6 | H3L2 | n2 | 324C6 | H2L1 | 4-4-20 | — |
| | | | | | | | | | (FITC) | |
| 35 | E12 | 4-4-20 | — | PL230C6 | H3L2 | n2 | 324C6 | H2L1 | 480C8 | H1L1 |
| | | (FITC) | | | | | | | | |
| 35 | E13 | 460C3 | L1H1 | PL230C6 | H3L2 | n2 | 4-4-20 | — | 480C8 | H1L1 |
| | | | | | | | (FITC) | | | |
| 35 | E15 | 460C3 | L1H1 | 4-4-20 | — | n2 | 324C6 | H2L1 | 480C8 | H1L1 |
| | | | | (FITC) | | | | | | |
| 35 | E18 | 460C3 | L1H1 | PL230C6 | H3L2 | n2 | 323H7 | H4L1 | 284A10 | H1L1 |
| 35 | E19 | 420H5 | L3H3 | PL230C6 | H3L2 | n2 | 323H7 | H4L1 | 284A10 | H1L1 |
| 35 | E20 | 466F6 | L5H2 | PL230C6 | H3L2 | n2 | 323H7 | H4L1 | 284A10 | H1L1 |
| 35 | E21 | 460C3 | L1H1 | PL230C6 | H3L2 | n2 | 338H4 | H3L4 | 284A10 | H1L1 |
| 35 | E22 | 420H5 | L3H3 | PL230C6 | H3L2 | n2 | 338H4 | H3L4 | 284A10 | H1L1 |
| 35 | E23 | 466F6 | L5H2 | PL230C6 | H3L2 | n2 | 338H4 | H3L4 | 284A10 | H1L1 |
| 35 | E24 | 460C3 | L1H1 | PL230C6 | H3L2 | n2 | 330F11 | H1L1 | 284A10 | H1L1 |
| 35 | E25 | 420H5 | L3H3 | PL230C6 | H3L2 | n2 | 330F11 | H1L1 | 284A10 | H1L1 |
| 35 | E26 | 466F6 | L5H2 | PL230C6 | H3L2 | n2 | 330F11 | H1L1 | 284A10 | H1L1 |
| 35 | E36 | 4-4-20 | — | PL230C6 | H3L2 | n2 | 338H4 | H3L4 | 284A10 | H1L1 |
| | | (FITC) | | | | | | | | |
| 35 | E37 | 460C3 | L1H1 | 4-4-20 | — | n2 | 338H4 | H3L4 | 284A10 | H1L1 |
| | | | | (FITC) | | | | | | |
| 35 | E38 | 460C3 | L1H1 | PL230C6 | H3L2 | n2 | 4-4-20 | — | 284A10 | H1L1 |
| | | | | | | | (FITC) | | | |
| 35 | E39 | 460C3 | L1H1 | PL230C6 | H3L2 | n2 | 338H4 | H3L4 | 4-4-20 | — |
| | | | | | | | | | (FITC) | |

### Example 8: FACS Analysis of Tetra-Specific Specific Antibody Binding to Human PD-L1 Transfected CHO Cells

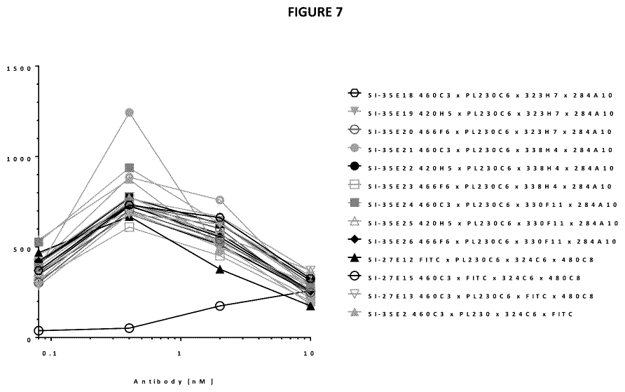

The tetra-specific-specific antibodies listed in TABLE 6 were tested for binding to Chinese hamster ovary cells (CHO) cells stably expressing full length human ROR1. Antibodies were prepared at 2× final concentration and titrated 1:5 across 3 wells of a 96 well plate in 50 ul of PBS/2% FBS and then 5,000 ROR1-CHO cells in 50 ul PBS/2% FBS were added. This mixture was incubated for 30 minutes on ice, washed once with 200 ul PBS/2% FBS, and then the secondary antibody PE Goat anti-Human IgG Fc at 1:1000 dilution of stock was added, and this mixture was incubated for 30 minutes on ice. Cells were washed 2×200 ul PBS/2% FBS, resuspended in 50 ul PBS/2% FBS and analyzed on a BD LSRFORTESSA® and the binding profile is shown in FIG. 7. All of the tetra-specific antibodies except for the control SI-27E15 contain the same PD-L1 binding domain, PL230C6, and showed very similar binding intensity to PD-L1 expressing CHO cells.

### Example 9: Re-Directed T Cell Cytotoxicity (RTCC) Assay with Peripheral Blood Mononuclear Cells as Effectors and B-Acute Lymphoblastic Leukemia (B-ALL) Cell Line Kasumi-2 as Targets

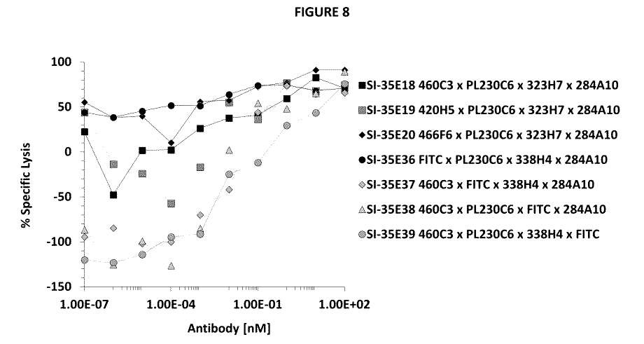

The tetra-specific-specific antibodies listed in TABLE 6 were tested for RTCC activity against the B-ALL cell line Kasumi 2 using human peripheral blood mononuclear cells (PBMC) as effectors. The Kasumi 2 target cells, 5×10e6, were labeled with CFSE (Invitrogen, #C34554) at 0.5 uM in 10 ml of culture media for 20 minutes at 37 C. The cells were washed 3 times with 50 ml of culture media before resuspending in 10 ml then counted again. Antibodies were prepared at 2× final concentration and titrated 1:3 across 10 wells of a 96 well plate in 200 ul of RPMI+10% FBS. Human PBMC were purified by standard ficoll density gradient from a “leukopak” which is an enriched leukapheresis product collected from normal human peripheral blood. In the final destination 96 well plate the target cells, PBMC, and serially titrated antibodies were combined by adding 100 ul of target cells (5,000), 50 ul of PBMC (25,000), and 100 ul of each antibody dilution to each well of the assay. The assay plate was incubated at 37 C for approximately 72 hours and then the contents of each assay well were harvested and analyzed for the number of CFSE-labeled target cells remaining. As shown on FIG. 8, the tetra-specific antibodies all contain the same PD-L1 binding domain PL230C6, the same ROR1 binding domain 323H7, and the same CD3 binding domain 284A10, but have one of the 41BB binding domains 460C3, 420H5, and 466F6 and showed greater RTCC activity compared to the controls except for the control SI-27E12 which does not have a 41BB binding domain but appeared to be similarly potent at the tetra-specific antibodies SI-35E18, 19, and 20.

### Example 10: Re-Directed T Cell Cytotoxicity (RTCC) Assay with CD8+, CD45RO+ Memory T Cells as Effectors and B-Acute Lymphoblastic Leukemia (B-ALL) Cell Line Kasumi-2 as Targets

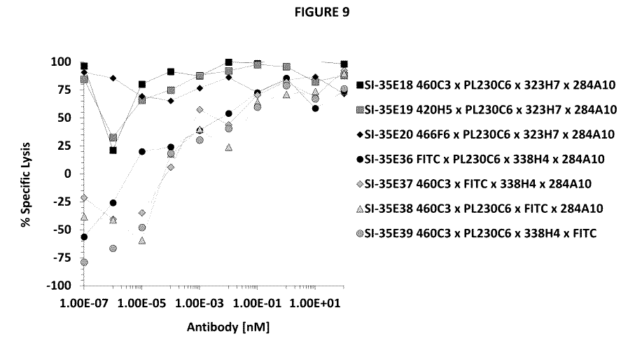

The tetra-specific-specific antibodies listed in TABLE 6 were tested for RTCC activity against the B-ALL cell line Kasumi 2 using human CD8+, CD45RO+ memory T cells as effectors. The Kasumi 2 target cells, 5×10e6, were labeled with CFSE (Invitrogen, #C34554) at 0.5 uM in 10 ml of culture media for 20 minutes at 37 C. The cells were washed 3 times with 50 ml of culture media before resuspending in 10 ml then counted again. Antibodies were prepared at 2× final concentration and titrated 1:3 across 10 wells of a 96 well plate in 200 ul of RPMI+10% FBS. Human CD8+, CD45RO+ memory T cells were enriched from peripheral blood mononuclear cells from a normal donor using the EASYSEP™ Human Memory CD8+ T Cell Enrichment Kit (Stemcell Technologies, #19159) as per the manufacturers protocol. The final cell population was determined to be 98% CD8+, CD45RO+ T cells by FACS analysis (data not shown). In the final destination 96 well plate the target cells, T cells, and serially titrated antibodies were combined by adding 100 ul of target cells (5,000), 50 ul of CD8+, CD45RO+ memory T cells (25,000), and 100 ul of each antibody dilution to each well of the assay. The assay plate was incubated at 37 C for approximately 72 hours and then the contents of each assay well were harvested and analyzed for the number of CFSE-labeled target cells remaining. As shown on FIG. 9, the tetra-specific antibodies all contain the same PD-L1 binding domain PL230C6, the same ROR1 binding domain 323H7, and the same CD3 binding domain 284A10, but have one of the 41BB binding domains 460C3, 420H5, and 466F6 and showed greater RTCC activity compared to the controls that do not contain one of the 41BB, PD-L1, ROR1, or CD3 binding domains.

### Example 11: Re-Directed T Cell Cytotoxicity (RTCC) Assay with CD8+, CD45RA+ Naive T Cells as Effectors and B-Acute Lymphoblastic Leukemia (B-ALL) Cell Line Kasumi-2 as Targets

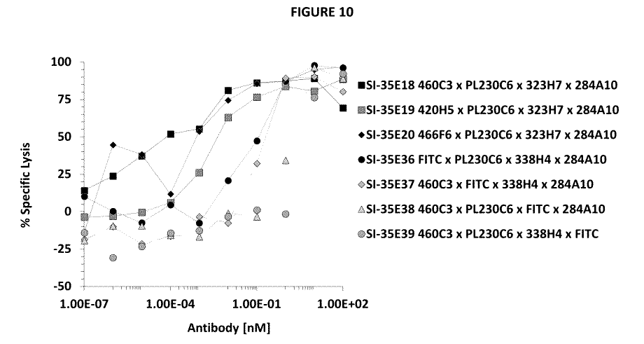

The tetra-specific-specific antibodies listed in TABLE 6 were tested for RTCC activity against the B-ALL cell line Kasumi 2 using human CD8+, CD45RA+ memory T cells as effectors. The Kasumi 2 target cells, 5×10e6, were labeled with CFSE (Invitrogen, #C34554) at 0.5 uM in 10 ml of culture media for 20 minutes at 37 C. The cells were washed 3 times with 50 ml of culture media before resuspending in 10 ml then counted again. Antibodies were prepared at 2× final concentration and titrated 1:3 across 10 wells of a 96 well plate in 200 ul of RPMI+10% FBS. Human CD8+, CD45RA+ memory T cells were enriched from peripheral blood mononuclear cells from a normal donor using the EASYSEP™ Human Naïve CD8+ T Cell Isolation Kit (Stemcell Technologies, #19258) as per the manufacturers protocol. The final cell population was determined to be 98% CD8+, CD45RA+ T cells by FACS analysis (data not shown). In the final destination 96 well plate the target cells, T cells, and serially titrated antibodies were combined by adding 100 ul of target cells (5,000), 50 ul of CD8+, CD45RO+ T cells (25,000), and 100 ul of each antibody dilution to each well of the assay. The assay plate was incubated at 37 C for approximately 72 hours and then the contents of each assay well were harvested and analyzed for the number of CFSE-labeled target cells remaining. As shown on FIG. 10, the tetra-specific antibodies all contain the same PD-L1 binding domain PL230C6, the same ROR1 binding domain 323H7, and the same CD3 binding domain 284A10, but have one of the 41BB binding domains 460C3, 420H5, and 466F6 and showed greater RTCC activity compared to the controls that do not contain one of the 41BB, PD-L1, ROR1, or CD3 binding domains.

### Example 12: Re-Directed T Cell Cytotoxicity (RTCC) Assay with Peripheral Blood Mononuclear Cells as Effectors and B-Acute Lymphoblastic Leukemia (B-ALL) Cell Line Kasumi-2 as Targets

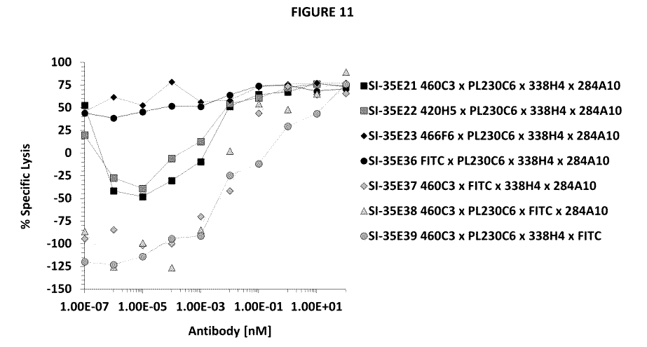

The tetra-specific-specific antibodies listed in TABLE 6 were tested for RTCC activity against the B-ALL cell line Kasumi 2 using human peripheral blood mononuclear cells (PBMC) as effectors. The Kasumi 2 target cells, 5×10e6, were labeled with CFSE (Invitrogen, #C34554) at 0.5 uM in 10 ml of culture media for 20 minutes at 37 C. The cells were washed 3 times with 50 ml of culture media before resuspending in 10 ml then counted again. Antibodies were prepared at 2× final concentration and titrated 1:3 across 10 wells of a 96 well plate in 200 ul of RPMI+10% FBS. Human PBMC were purified by standard ficoll density gradient from a “leukopak” which is an enriched leukapheresis product collected from normal human peripheral blood. In the final destination 96 well plate the target cells, PBMC, and serially titrated antibodies were combined by adding 100 ul of target cells (5,000), 50 ul of PBMC (25,000), and 100 ul of each antibody dilution to each well of the assay. The assay plate was incubated at 37 C for approximately 72 hours and then the contents of each assay well were harvested and analyzed for the number of CFSE-labeled target cells remaining. As shown on FIG. 11, the tetra-specific antibodies all contain the same PD-L1 binding domain PL230C6, the same ROR1 binding domain 338H4, and the same CD3 binding domain 284A10, but have one of the 41BB binding domains 460C3, 420H5, and 466F6 and showed greater RTCC activity compared to the controls except for the control SI-35E36 which does not have a 41BB binding domain but appeared to be similarly potent at the tetra-specific antibodies SI-35E18, 19, and 20.

### Example 13: Re-Directed T Cell Cytotoxicity (RTCC) Assay with CD8+, CD45RO+ Memory T Cells as Effectors and B-Acute Lymphoblastic Leukemia (B-ALL) Cell Line Kasumi-2 as Targets

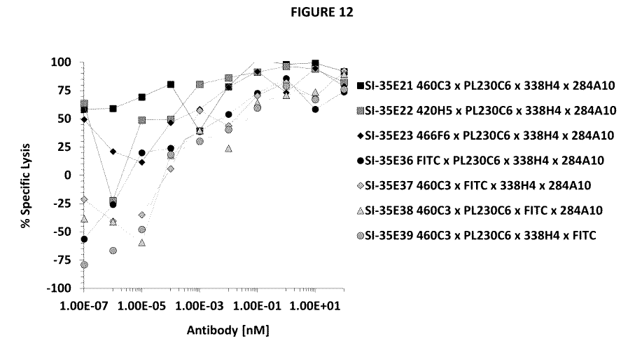

The tetra-specific-specific antibodies listed in TABLE 6 were tested for RTCC activity against the B-ALL cell line Kasumi 2 using human CD8+, CD45RO+ memory T cells as effectors. The Kasumi 2 target cells, 5×10e6, were labeled with CFSE (Invitrogen, #C34554) at 0.5 uM in 10 ml of culture media for 20 minutes at 37 C. The cells were washed 3 times with 50 ml of culture media before resuspending in 10 ml then counted again. Antibodies were prepared at 2× final concentration and titrated 1:3 across 10 wells of a 96 well plate in 200 ul of RPMI+10% FBS. Human CD8+, CD45RO+ memory T cells were enriched from peripheral blood mononuclear cells from a normal donor using the EASYSEP™ Human Memory CD8+ T Cell Enrichment Kit (Stemcell Technologies, #19159) as per the manufacturers protocol. The final cell population was determined to be 98% CD8+, CD45RO+ T cells by FACS analysis (data not shown). In the final destination 96 well plate the target cells, T cells, and serially titrated antibodies were combined by adding 100 ul of target cells (5,000), 50 ul of CD8+, CD45RO+ memory T cells (25,000), and 100 ul of each antibody dilution to each well of the assay. The assay plate was incubated at 37 C for approximately 72 hours and then the contents of each assay well were harvested and analyzed for the number of CFSE-labeled target cells remaining. As shown on FIG. 12, the tetra-specific antibodies all contain the same PD-L1 binding domain PL230C6, the same ROR1 binding domain 338H4, and the same CD3 binding domain 284A10, but have one of the 41BB binding domains 460C3, 420H5, and 466F6 and showed greater RTCC activity compared to the controls that do not contain one of the 41BB, PD-L1, ROR1, or CD3 binding domains.

### Example 14: Re-Directed T Cell Cytotoxicity (RTCC) Assay with CD8+, CD45RA+ Naive T Cells as Effectors and B-Acute Lymphoblastic Leukemia (B-ALL) Cell Line Kasumi-2 as Targets

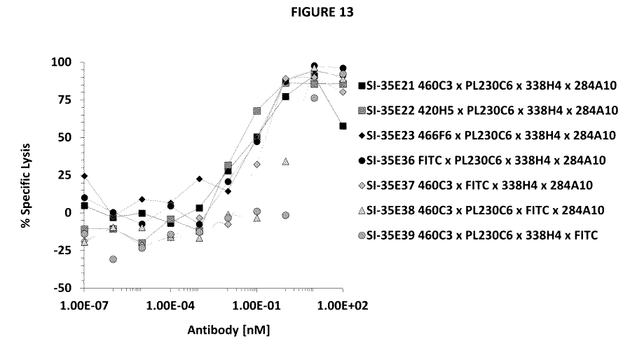

The tetra-specific-specific antibodies listed in TABLE 6 were tested for RTCC activity against the B-ALL cell line Kasumi 2 using human CD8+, CD45RA+ memory T cells as effectors. The Kasumi 2 target cells, 5×10e6, were labeled with CFSE (Invitrogen, #C34554) at 0.5 uM in 10 ml of culture media for 20 minutes at 37 C. The cells were washed 3 times with 50 ml of culture media before resuspending in 10 ml then counted again. Antibodies were prepared at 2× final concentration and titrated 1:3 across 10 wells of a 96 well plate in 200 ul of RPMI+10% FBS. Human CD8+, CD45RA+ memory T cells were enriched from peripheral blood mononuclear cells from a normal donor using the EASYSEP™ Human Naïve CD8+ T Cell Isolation Kit (Stemcell Technologies, #19258) as per the manufacturers protocol. The final cell population was determined to be 98% CD8+, CD45RA+ T cells by FACS analysis (data not shown). In the final destination 96 well plate the target cells, T cells, and serially titrated antibodies were combined by adding 100 ul of target cells (5,000), 50 ul of CD8+, CD45RO+ T cells (25,000), and 100 ul of each antibody dilution to each well of the assay. The assay plate was incubated at 37 C for approximately 72 hours and then the contents of each assay well were harvested and analyzed for the number of CFSE-labeled target cells remaining. As shown on FIG. 13, the tetra-specific antibodies all contain the same PD-L1 binding domain PL230C6, the same ROR1 binding domain 338H4, and the same CD3 binding domain 284A10, but have one of the 41BB binding domains 460C3, 420H5, and 466F6 but did not show greater RTCC activity compared to the controls that do not contain one of the 41BB, PD-L1, ROR1, or CD3 binding domains. This is in contrast to the tetra-specific antibodies described in example 6 and shown in FIG. 10 that do show RTCC activity with CD8+, CD45RA+naïve T cells.

While the present disclosure has been described with reference to particular embodiments or examples, it may be understood that the embodiments are illustrative and that the disclosure scope is not so limited. Alternative embodiments of the present disclosure may become apparent to those having ordinary skill in the art to which the present disclosure pertains. Such alternate embodiments are considered to be encompassed within the scope of the present disclosure. Accordingly, the scope of the present disclosure is defined by the appended claims and is supported by the foregoing description. All references cited or referred to in this disclosure are hereby incorporated by reference in their entireties.

### Multi-Specific Antibodies and Methods of Making and Using Thereof

SEQUENCE LISTING
CDR's underlined in amino acid sequences
anti-CD3 284A10 VHv1 nt
SEQ ID: 01
GAGGTGCAGCTGGTGGAGTCTGGGGGAGGCTTGGTCCAGCCTGGG
GGGTCCCTGAGACTCTCCTGTGCAGCCTCTGGATTCACCATCAGT
ACCAATGCAATGAGCTGGGTCCGCCAGGCTCCAGGGAAGGGGCTG
GAGTGGATCGGAGTCATTACTGGTCGTGATATCACATACTACGCG
AGCTGGGCGAAAGGCAGATTCACCATCTCCAGAGACAATTCCAAG
AACACGCTGTATCTTCAAATGAACAGCCTGAGAGCCGAGGACACG
GCTGTGTATTACTGTGCGCGCGACGGTGGATCATCTGCTATTACT
AGTAACAACATTTGGGGCCAAGGAACTCTGGTCACCGTTTCTTCA
anti-CD3 284A10 VHv1 aa
SEQ ID: 02
EVQLVESGGGLVQPGGSLRLSCAASGFTISTNAMSWVRQAPGKGL
EWIGVITGRDITYYASWAKGRFTISRDNSKNTLYLQMNSLRAEDT
AVYYCARDGGSSAITSNNIWGQGTLVTVSS
anti-CD3 284A10 VLv1 nt
SEQ ID: 03
GACGTCGTGATGACCCAGTCTCCTTCCACCCTGTCTGCATCTGTA
GGAGACAGAGTCACCATCAATTGCCAAGCCAGTGAGAGCATTAGC
AGTTGGTTAGCCTGGTATCAGCAGAAACCAGGGAAAGCCCCTAAG
CTCCTGATCTATGAAGCATCCAAACTGGCATCTGGGGTCCCATCA
AGGTTCAGCGGCAGTGGATCTGGGACAGAGTTCACTCTCACCATC
AGCAGCCTGCAGCCTGATGATTTTGCAACTTATTACTGCCAAGGC
TATTTTTATTTTATTAGTCGTACTTATGTAAATTCTTTCGGCGGA
GGGACCAAGGTGGAGATCAAA
anti-CD3 284A10 VLv1 aa
SEQ ID: 04
DVVMTQSPSTLSASVGDRVTINCQASESISSWLAWYQQKPGKAPK
LLIYEASKLASGVPSRFSGSGSGTEFTLTISSLQPDDFATYYCQG
YFYFISRTYVNSFGGGTKVEIK
anti-CD3 480C8 VHv1 nt
SEQ ID: 05
GAGGTGCAGCTGGTGGAGTCTGGGGGAGGCTTGGTCCAGCCTGGG
GGGTCCCTGAGACTCTCCTGTGCAGCCTCTGGAATCGACCTCAGT
AGCAATGCAATGAGCTGGGTCCGCCAGGCTCCAGGGAAGGGGCTG
GAGTGGATCGGAGTCATTACTGGTCGTGATATCACATACTACGCG
AGCTGGGCGAAAGGCAGATTCACCATCTCCAGAGACAATTCCAAG
AACACGCTGTATCTTCAAATGAACAGCCTGAGAGCCGAGGACACG
GCTGTGTATTACTGTGCGCGCGACGGTGGATCATCTGCTATTAAT
AGTAAGAACATTTGGGGCCAAGGAACTCTGGTCACCGTTTCTTCA
anti-CD3 480C8 VHv1 aa
SEQ ID: 06
EVQLVESGGGLVQPGGSLRLSCAASGIDLSSNAMSWVRQAPGKGL
EWIGVITGRDITYYASWAKGRFTISRDNSKNTLYLQMNSLRAEDT
AVYYCARDGGSSAINSKNIWGQGTLVTVSS
anti-CD3 480C8 VLv1 nt
SEQ ID: 07
GACATCCAGATGACCCAGTCTCCTTCCACCCTGTCTGCATCTGTA
GGAGACAGAGTCACCATCACTTGCCAAGCCAGTGAGAGCATTAGC
AGTTGGTTAGCCTGGTATCAGCAGAAACCAGGGAAAGCCCCTAAG
CTCCTGATCTATGAAGCATCCAAACTGGCATCTGGGGTCCCATCA
AGGTTCAGCGGCAGTGGATCTGGGACAGAGTTCACTCTCACCATC
AGCAGCCTGCAGCCTGATGATTTTGCAACTTATTACTGCCAAGGC
TATTTTTATTTTATTAGTCGTACTTATGTAAATGCTTTCGGCGGA
GGGACCAAGGTGGAGATCAAA
anti-CD3 480C8 VLv1 aa
SEQ ID: 08
DIQMTQSPSTLSASVGDRVTITCQASESISSWLAWYQQKPGKAPK
LLIYEASKLASGVPSRFSGSGSGTEFTLTISSLQPDDFATYYCQG
YFYFISRTYVNAFGGGTKVEIK
anti-PD-L1 PL230C6 VHv3 nt
SEQ ID: 09
CAGTCGGTGGAGGAGTCTGGGGGAGGCTTGGTCCAGCCTGGGGGG
TCCCTGAGACTCTCCTGTACAGCCTCTGGAATCGACCTTAATACC
TACGACATGATCTGGGTCCGCCAGGCTCCAGGCAAGGGGCTAGAG
TGGGTTGGAATCATTACTTATAGTGGTAGTAGATACTACGCGAAC
TGGGCGAAAGGCCGATTCACCATCTCCAAAGACAATACCAAGAAC
ACGGTGTATCTGCAAATGAACAGCCTGAGAGCTGAGGACACGGCT
GTGTATTACTGTGCCAGAGATTATATGAGTGGTTCCCACTTGTGG
GGCCAGGGAACCCTGGTCACCGTCTCTAGT
anti-PD-L1 PL230C6 VHv3 aa
SEQ ID: 10
QSVEESGGGLVQPGGSLRLSCTASGIDLNTYDMIWVRQAPGKGLE
WVGIITYSGSRYYANWAKGRFTISKDNTKNTVYLQMNSLRAEDTA
VYYCARDYMSGSHLWGQGTLVTVSS
anti-PD-L1 PL230C6 VLv2 nt
SEQ ID: 11
GCCTATGATATGACCCAGTCTCCATCTTCCGTGTCTGCATCTGTA
GGAGACAGAGTCACCATCAAGTGTCAGGCCAGTGAGGACATTTAT
AGCTTCTTGGCCTGGTATCAGCAGAAACCAGGGAAAGCCCCTAAG
CTCCTGATCCATTCTGCATCCTCTCTGGCATCTGGGGTCCCATCA
AGGTTCAGCGGCAGTGGATCTGGGACAGATTTCACTCTCACCATC
AGCAGCCTGCAGCCTGAAGATTTTGCAACTTACTATTGTCAACAG
GGTTATGGTAAAAATAATGTTGATAATGCTTTCGGCGGAGGGACC
AAGGTGGAGATCAAA
anti-PD-L1 PL230C6 VLv2 aa
SEQ ID: 12
AYDMTQSPSSVSASVGDRVTIKCQASEDIYSFLAWYQQKPGKAPK
LLIHSASSLASGVPSRFSGSGSGTDFTLTISSLQPEDFATYYCQQ
GYGKNNVDNAFGGGTKVEIK
anti-PD-L1 PL221G5 VHv1 nt
SEQ ID: 13
GAGGTGCAGCTGTTGGAGTCTGGGGGAGGCTTGGTACAGCCTGGG
GGGTCCCTGAGACTCTCCTGTGCAGCCTCTGGATTCTCCTTCAGT
AGCGGGTACGACATGTGCTGGGTCCGCCAGGCTCCAGGGAAGGGG
CTGGAGTGGATCGCATGCATTGCTGCTGGTAGTGCTGGTATCACT
TACGACGCGAACTGGGCGAAAGGCCGGTTCACCATCTCCAGAGAC
AATTCCAAGAACACGCTGTATCTGCAAATGAACAGCCTGAGAGCC
GAGGACACGGCCGTATATTACTGTGCGAGATCGGCGTTTTCGTTC
GACTACGCCATGGACCTCTGGGGCCAGGGAACCCTGGTCACCGTC
TCGAGC
anti-PD-L1 PL221G5 VHv1 aa
SEQ ID: 14
EVQLLESGGGLVQPGGSLRLSCAASGFSFSSGYDMCWVRQAPGKG
LEWIACIAAGSAGITYDANWAKGRFTISRDNSKNTLYLQMNSLRA
EDTAVYYCARSAFSFDYAMDLWGQGTLVTVSS
anti-PD-L1 PL221G5 VLv1 nt
SEQ ID: 15
GACATCCAGATGACCCAGTCTCCTTCCACCCTGTCTGCATCTGTA
GGAGACAGAGTCACCATCACTTGCCAGGCCAGTCAGAGCATTAGT
TCCCACTTAAACTGGTATCAGCAGAAACCAGGGAAAGCCCCTAAG
CTCCTGATCTATAAGGCATCCACTCTGGCATCTGGGGTCCCATCA
AGGTTCAGCGGCAGTGGATCTGGGACAGAATTTACTCTCACCATC
AGCAGCCTGCAGCCTGATGATTTTGCAACTTATTACTGCCAACAG
GGTTATAGTTGGGGTAATGTTGATAATGTTTTCGGCGGAGGGACC
AAGGTGGAGATCAAA
anti-PD-L1 PL221G5 VLv1 aa
SEQ ID: 16
DIQMTQSPSTLSASVGDRVTITCQASQSISSHLNWYQQKPGKAPK
LLIYKASTLASGVPSRFSGSGSGTEFTLTISSLQPDDFATYYCQQ
GYSWGNVDNVFGGGTKVEIK
anti-PD-1 PD224D1 VHv2 nt
SEQ ID: 17
GAGGTGCAGCTGGTGGAGTCTGGGGGAGGCTTGGTCCAGCCTGGG
GGGTCCCTGAGACTCTCCTGTACAGCCTCTGGATTCTCCCTAAGT
AGCTATGCAATGAGCTGGGTCCGCCAGGCTCCAGGGAAGGGGCTG
GAGTACATCGGCTACATTGGTGATACTACTGGCATAGCCTACGCG
AGCTGGGCGAATGGCAGATTCACCATCTCCAAAGACAATACCAAG
AACACGGTGGATCTTCAAATGAACAGCCTGAGAGCCGAGGACACG
GCTGTGTATTACTGTGCGAGAGGCTGGTCCTACTTAGACATCTGG
GGCCAAGGGACCCTGGTCACCGTCTCGAGC
anti-PD-1 PD224D1 VHv2 aa
SEQ ID: 18
EVQLVESGGGLVQPGGSLRLSCTASGFSLSSYAMSWVRQAPGKGL
EYIGYIGDTTGIAYASWANGRFTISKDNTKNTVDLQMNSLRAEDT
AVYYCARGWSYLDIWGQGTLVTVSS
anti-PD-1 PD224D1 VLv2 nt
SEQ ID: 19
GCCCTTGTGATGACCCAGTCTCCATCCTCCCTGTCTGCATCTGTA
GGAGACAGAGTCACCATCACTTGCCAGGCCAGTCAGAACATTTAC
AGCAATTTAGCCTGGTATCAGCAGAAACCAGGGAAAGTTCCTAAG
CTCCTGATCTATCAGGCCTCCACTCTGGCATCTGGGGTCCCATCT
CGGTTCAGTGGCAGTGGATATGGGACAGATTTCACTCTCACCATC
AGCAGCCTGCAGCCTGAAGATGTTGCAACTTATTACTGTCAAGGC
GGTTATTATAGTGCTGCCCTTAATACTTTCGGCGGAGGGACCAAG
GTGGAGATCAAA
anti-PD-1 PD224D1 VLv2 aa
SEQ ID: 20
ALVMTQSPSSLSASVGDRVTITCQASQNIYSNLAWYQQKPGKVPK
LLIYQASTLASGVPSRFSGSGYGTDFTLTISSLQPEDVATYYCQG
GYYSAALNTFGGGTKVEIK
anti-4-1BB 420H5 VHv3 nt
SEQ ID: 21
CAGTCGCTGGTGGAGTCTGGGGGAGGCTTGGTACAGCCTGGGGGG
TCCCTGAGACTCTCCTGTGCAGCCTCTGGATTCTCCTTCAGTAGC
AACTACTGGATATGCTGGGTCCGCCAGGCTCCAGGGAAGGGGCTG
GAGTGGATCGCATGCATTTATGTTGGTAGTAGTGGTGACACTTAC
TACGCGAGCTCCGCGAAAGGCCGGTTCACCATCTCCAGAGACAAT
TCCAAGAACACGCTGTATCTGCAAATGAACAGCCTGAGAGCCGAG
GACACGGCCGTATATTACTGTGCGAGAGATAGTAGTAGTTATTAT
ATGTTTAACTTGTGGGGCCAGGGAACCCTGGTCACCGTCTCGAGC
anti-4-1BB 420H5 VHv3 aa
SEQ ID: 22
QSLVESGGGLVQPGGSLRLSCAASGFSFSSNYWICWVRQAPGKGL
EWIACIYVGSSGDTYYASSAKGRFTISRDNSKNTLYLQMNSLRAE
DTAVYYCARDSSSYYMFNLWGQGTLVTVSS
anti-4-1BB 420H5 VLv3 nt
SEQ ID: 23
GCCCTTGTGATGACCCAGTCTCCTTCCACCCTGTCTGCATCTGTA
GGAGACAGAGTCACCATCAATTGCCAGGCCAGTGAGGACATTGAT
ACCTATTTAGCCTGGTATCAGCAGAAACCAGGGAAAGCCCCTAAG
CTCCTGATCTTTTATGCATCCGATCTGGCATCTGGGGTCCCATCA
AGGTTCAGCGGCAGTGGATCTGGGACAGAATTCACTCTCACCATC
AGCAGCCTGCAGCCTGATGATTTTGCAACTTATTACTGCCAAGGC
GGTTACTATACTAGTAGTGCTGATACGAGGGGTGCTTTCGGCGGA
GGGACCAAGGTGGAGATCAAA
anti-4-1BB 420H5 VLv3 aa
SEQ ID: 24
ALVMTQSPSTLSASVGDRVTINCQASEDIDTYLAWYQQKPGKAPK
LLIFYASDLASGVPSRFSGSGSGTEFTLTISSLQPDDFATYYCQG
GYYTSSADTRGAFGGGTKVEIK
anti-4-1BB 466F6 VHv2 nt
SEQ ID: 25
CGGTCGCTGGTGGAGTCTGGGGGAGGCTTGGTCCAGCCTGGGGGG
TCCCTGAGACTCTCCTGTACAGCCTCTGGATTCACCATCAGTAGC
TACCACATGCAGTGGGTCCGCCAGGCTCCAGGGAAGGGGCTGGAG
TACATCGGAACCATTAGTAGTGGTGGTAATGTATACTACGCGAGC
TCCGCGAGAGGCAGATTCACCATCTCCAGACCCTCGTCCAAGAAC
ACGGTGGATCTTCAAATGAACAGCCTGAGAGCCGAGGACACGGCT
GTGTATTACTGTGCGAGAGACTCTGGTTATAGTGATCCTATGTGG
GGCCAGGGAACCCTGGTCACCGTCTCGAGC
anti-4-1BB 466F6 VHv2 aa
SEQ ID: 26
RSLVESGGGLVQPGGSLRLSCTASGFTISSYHMQWVRQAPGKGLE
YIGTISSGGNVYYASSARGRFTISRPSSKNTVDLQMNSLRAEDTA
VYYCARDSGYSDPMWGQGTLVTVSS
anti-4-1BB 466F6 VLv5 nt
SEQ ID: 27
GACGTTGTGATGACCCAGTCTCCATCTTCCGTGTCTGCATCTGTA
GGAGACAGAGTCACCATCACCTGTCAGGCCAGTCAGAACATTAGG
ACTTACTTATCCTGGTATCAGCAGAAACCAGGGAAAGCCCCTAAG
CTCCTGATCTATGCTGCAGCCAATCTGGCATCTGGGGTCCCATCA
AGGTTCAGCGGCAGTGGATCTGGGACAGATTTCACTCTCACCATC
AGCGACCTGGAGCCTGGCGATGCTGCAACTTACTATTGTCAGTCT
ACCTATCTTGGTACTGATTATGTTGGCGGTGCTTTCGGCGGAGGG
ACCAAGGTGGAGATCAAA
anti-4-1BB 466F6 VLv5 aa
SEQ ID: 28
DVVMTQSPSSVSASVGDRVTITCQASQNIRTYLSWYQQKPGKAPK
LLIYAAANLASGVPSRFSGSGSGTDFTLTISDLEPGDAATYYCQS
TYLGTDYVGGAFGGGTKVEIK
anti-4-1BB 460C3 VHv1 nt
SEQ ID: 29
GAGGTGCAGCTGTTGGAGTCTGGGGGAGGCTTGGTACAGCCTGGG
GGGTCCCTGAGACTCTCCTGTGCAGCCTCTGGAATCGACTTCAGT
AGGAGATACTACATGTGCTGGGTCCGCCAGGCTCCAGGGAAGGGG
CTGGAGTGGATCGCATGCATATATACTGGTAGCCGCGATACTCCT
CACTACGCGAGCTCCGCGAAAGGCCGGTTCACCATCTCCAGAGAC
AATTCCAAGAACACGCTGTATCTGCAAATGAACAGCCTGAGAGCC
GAGGACACGGCCGTATATTACTGTGCGAGAGAAGGTAGCCTGTGG
GGCCAGGGAACCCTGGTCACCGTCTCGAGC
anti-4-1BB 460C3 VHv1 aa
SEQ ID: 30
EVQLLESGGGLVQPGGSLRLSCAASGIDFSRRYYMCWVRQAPGKG
LEWIACIYTGSRDTPHYASSAKGRFTISRDNSKNTLYLQMNSLRA
EDTAVYYCAREGSLWGQGTLVTVSS
anti-4-1BB 460C3 VLv1 nt
SEQ ID: 31
GACATCCAGATGACCCAGTCTCCTTCCACCCTGTCTGCATCTGTA
GGAGACAGAGTCACCATCACTTGCCAGTCCAGTCAGAGTGTTTAT
AGTAACTGGTTCTCCTGGTATCAGCAGAAACCAGGGAAAGCCCCT
AAGCTCCTGATCTATTCTGCATCCACTCTGGCATCTGGGGTCCCA
TCAAGGTTCAGCGGCAGTGGATCTGGGACAGAATTCACTCTCACC
ATCAGCAGCCTGCAGCCTGATGATTTTGCAACTTATTACTGCGCA
GGCGGTTACAATACTGTTATTGATACTTTTGCTTTCGGCGGAGGG
ACCAAGGTGGAGATCAAA
anti-4-1BB 460C3 VLv1 aa
SEQ ID: 32
DIQMTQSPSTLSASVGDRVTITCQSSQSVYSNWFSWYQQKPGKAP
KLLIYSASTLASGVPSRFSGSGSGTEFTLTISSLQPDDFATYYCA
GGYNTVIDTFAFGGGTKVEIK
anti-ROR1 324C6 VHv2 nt
SEQ ID: 33
CAGTCGCTGGTGGAGTCTGGGGGAGGCTTGGTCCAGCCTGGGGGG
TCCCTGAGACTCTCCTGTACTGCCTCTGGATTCTCCCTCAGTAGG
TACTACATGACCTGGGTCCGCCAGGCTCCAGGGAAGGGGCTGGAG
TGGATCGGAACCATTTATACTAGTGGTAGTACATGGTACGCGAGC
TGGACAAAAGGCAGATTCACCATCTCCAAAGACAATACCAAGAAC
ACGGTGGATCTTCAAATGAACAGCCTGAGAGCCGAGGACACGGCT
GTGTATTACTGTGCGAGATCCTATTATGGCGGTGATAAGACTGGT
TTAGGCATCTGGGGCCAGGGAACTCTGGTTACCGTCTCTTCA
anti-ROR1 324C6 VHv2 nt
SEQ ID: 34
QSLVESGGGLVQPGGSLRLSCTASGFSLSRYYMTWVRQAPGKGLE
WIGTIYTSGSTWYASWTKGRFTISKDNTKNTVDLQMNSLRAEDTA
VYYCARSYYGGDKTGLGIWGQGTLVTVSS
anti-ROR1 324C6 VLv1 nt
SEQ ID: 35
GACATCCAGATGACCCAGTCTCCTTCCACCCTGTCTGCATCTGTA
GGAGACAGAGTCACCATCACTTGCCAGGCCAGTCAGAGCATTGAT
AGTTGGTTATCCTGGTATCAGCAGAAACCAGGGAAAGCCCCTAAG
CTCCTGATCTATCAGGCATCCACTCTGGCATCTGGGGTCCCATCA
AGGTTCAGCGGCAGTGGATCTGGGACAGAGTTCACTCTCACCATC
AGCAGCCTGCAGCCTGATGATTTTGCAACTTATTACTGCCAATCT
GCTTATGGTGTTAGTGGTACTAGTAGTTATTTATATACTTTCGGC
GGAGGGACCAAGGTGGAGATCAAA
anti-ROR1 324C6 VLv1 aa
SEQ ID: 36
DIQMTQSPSTLSASVGDRVTITCQASQSIDSWLSWYQQKPGKAPK
LLIYQASTLASGVPSRFSGSGSGTEFTLTISSLQPDDFATYYCQS
AYGVSGTSSYLYTFGGGTKVEIK
anti-ROR1 323H7 VHv4 nt
SEQ ID: 37
GAGGTGCAGCTGTTGGAGTCTGGGGGAGGCTTGGTACAGCCTGGG
GGGTCCCTGAGACTCTCCTGTGCAGCCTCTGGATTCACCATCAGT
CGCTACCACATGACTTGGGTCCGCCAGGCTCCAGGGAAGGGGCTG
GAGTGGATCGGACATATTTATGTTAATAATGATGACACAGACTAC
GCGAGCTCCGCGAAAGGCCGGTTCACCATCTCCAGAGACAATTCC
AAGAACACGCTGTATCTGCAAATGAACAGCCTGAGAGCCGAGGAC
ACGGCCACCTATTTCTGTGCGAGATTGGATGTTGGTGGTGGTGGT
GCTTATATTGGGGACATCTGGGGCCAGGGAACTCTGGTTACCGTC
TCTTCA
anti-ROR1 323H7 VHv4 aa
SEQ ID: 38
EVQLLESGGGLVQPGGSLRLSCAASGFTISRYHMTWVRQAPGKGL
EWIGHIYVNNDDTDYASSAKGRFTISRDNSKNTLYLQMNSLRAED
TATYFCARLDVGGGGAYIGDIWGQGTLVTVSS
anti-ROR1 323H7 VLv1 nt
SEQ ID: 39
GACATCCAGATGACCCAGTCTCCATCCTCCCTGTCTGCATCTGTA
GGAGACAGAGTCACCATCACTTGCCAGTCCAGTCAGAGTGTTTAT
AACAACAACGACTTAGCCTGGTATCAGCAGAAACCAGGGAAAGTT
CCTAAGCTCCTGATCTATTATGCTTCCACTCTGGCATCTGGGGTC
CCATCTCGGTTCAGTGGCAGTGGATCTGGGACAGATTTCACTCTC
ACCATCAGCAGCCTGCAGCCTGAAGATGTTGCAACTTATTACTGT
GCAGGCGGTTATGATACGGATGGTCTTGATACGTTTGCTTTCGGC
GGAGGGACCAAGGTGGAGATCAAA
anti-ROR1 323H7 VLv1 aa
SEQ ID: 40
DIQMTQSPSSLSASVGDRVTITCQSSQSVYNNNDLAWYQQKPGKV
PKLLIYYASTLASGVPSRFSGSGSGTDFTLTISSLQPEDVATYYC
AGGYDTDGLDTFAFGGGTKVEIK
anti-ROR1 338H4 VHv3 nt
SEQ ID: 41
GAGGTGCAGCTGGTGGAGTCTGGGGGAGGCTTGGTCCAGCCTGGG
GGGTCCCTGAGACTCTCCTGTACTGCCTCTGGATTCTCCCTCAGT
AGCTATGCAATGAGCTGGGTCCGCCAGGCTCCAGGGAGGGGGCTG
GAGTGGATCGGAATCATTTATGCTAGTGGTAGCACATACTACGCG
AGCTCGGCGAAAGGCAGATTCACCATCTCCAAAGACAATACCAAG
AACACGGTGGATCTTCAAATGAACAGCCTGAGAGCCGAGGACACG
GCTGTGTATTACTGTGCGAGAATTTATGACGGCATGGACCTCTGG
GGCCAGGGAACTCTGGTTACCGTCTCTTCA
anti-ROR1 338H4 VHv3 aa
SEQ ID: 42
EVQLVESGGGLVQPGGSLRLSCTASGFSLSSYAMSWVRQAPGRGL
EWIGIIYASGSTYYASSAKGRFTISKDNTKNTVDLQMNSLRAEDT
AVYYCARIYDGMDLWGQGTLVTVSS
anti-ROR1 338H4 VLv4 nt
SEQ ID: 43
GACATCCAGATGACCCAGTCTCCATCCTCCCTGTCTGCATCTGTA
GGAGACAGAGTCACCATCAATTGCCAGGCCAGTCAGAACATTTAC
AGCTACTTATCCTGGTATCAGCAGAAACCAGGGAAAGTTCCTAAG
CGCCTGATCTATCTGGCATCTACTCTGGCATCTGGGGTCCCATCT
CGGTTCAGTGGCAGTGGATCTGGGACAGATTACACTCTCACCATC
AGCAGCCTGCAGCCTGAAGATGTTGCAACTTATTACTGTCAAAGC
AATTATAACGGTAATTATGGTTTCGGCGGAGGGACCAAGGTGGAG
ATCAAA
anti-ROR1 338H4 VLv4 aa
SEQ ID: 44
DIQMTQSPSSLSASVGDRVTINCQASQNIYSYLSWYQQKPGKVPK
RLIYLASTLASGVPSRFSGSGSGTDYTLTISSLQPEDVATYYCQS
NYNGNYGFGGGTKVEIK
anti-ROR1 330F11 VHv1 nt
SEQ ID: 45
GAGGTGCAGCTGGTGGAGTCTGGGGGAGGCTTGGTCCAGCCTGGG
GGGTCCCTGAGACTCTCCTGTGCAGCCTCTGGATTCTCCCTCAAT
AACTACTGGATGAGCTGGGTCCGCCAGGCTCCAGGGAAGGGGCTG
GAGTGGATCGGAACCATTAGTAGTGGTGCGTATACATGGTTCGCC
ACCTGGGCGACAGGCAGATTCACCATCTCCAGAGACAATTCCAAG
AACACGCTGTATCTTCAAATGAACAGCCTGAGAGCCGAGGACACG
GCTGTGTATTACTGTGCGAGATATTCTTCTACTACTGATTGGACC
TACTTTAACATCTGGGGCCAGGGAACTCTGGTTACCGTCTCTTCA
anti-ROR1 330F11 VHv1 aa
SEQ ID: 46
EVQLVESGGGLVQPGGSLRLSCAASGFSLNNYWMSWVRQAPGKGL
EWIGTISSGAYTWFATWATGRFTISRDNSKNTLYLQMNSLRAEDT
AVYYCARYSSTTDWTYFNIWGQGTLVTVSS
anti-ROR1 330F11 VLv1 nt
SEQ ID: 47
GACATCCAGATGACCCAGTCTCCTTCCACCCTGTCTGCATCTGTA
GGAGACAGAGTCACCATCACTTGCCAGGCCAGTCAGAGCATTAAT
AACTACTTAGCCTGGTATCAGCAGAAACCAGGGAAAGCCCCTAAG
CTCCTGATCTATAGGGCATCCACTCTGGAATCTGGGGTCCCATCA
AGGTTCAGCGGCAGTGGATCTGGGACAGAATTCACTCTCACCATC
AGCAGCCTGCAGCCTGATGATTTTGCAACTTATTACTGCCAAAGC
TATAATGGTGTTGGTAGGACTGCTTTCGGCGGAGGGACCAAGGTG
GAGATCAAA
anti-ROR1 330F11 VLv1 aa
SEQ ID: 48
DIQMTQSPSTLSASVGDRVTITCQASQSINNYLAWYQQKPGKAPK
LLIYRASTLESGVPSRFSGSGSGTEFTLTISSLQPDDFATYYCQS
YNGVGRTAFGGGTKVEIK
anti-EGFRvIII mAb 806 VH nt
SEQ ID: 49
GATGTGCAGCTTCAGGAGTCGGGACCTAGCCTGGTGAAACCTTCT
CAGTCTCTGTCCCTCACCTGCACTGTCACTGGCTACTCAATCACC
AGTGATTTTGCCTGGAACTGGATTCGGCAGTTTCCAGGAAACAAG
CTGGAGTGGATGGGCTACATAAGTTATAGTGGTAACACTAGGTAC
AACCCATCTCTCAAAAGTCGAATCTCTATCACTCGCGACACATCC
AAGAACCAATTCTTCCTGCAGTTGAACTCTGTGACTATTGAGGAC
ACAGCCACATATTACTGTGTAACGGCGGGACGCGGGTTTCCTTAT
TGGGGCCAAGGGACTCTGGTCACTGTCTCTGCA
anti-EGFRvIII mAb 806 VH aa
SEQ ID: 50
DVQLQESGPSLVKPSQSLSLTCTVTGYSITSDFAWNWIRQFPGNK
LEWMGYISYSGNTRYNPSLKSRISITRDTSKNQFFLQLNSVTIED
TATYYCVTAGRGFPYWGQGTLVTVSA
anti-EGFRvIII mAb 806 VL nt
SEQ ID: 51
GACATCCTGATGACCCAATCTCCATCCTCCATGTCTGTATCTCTG
GGAGACACAGTCAGCATCACTTGCCATTCAAGTCAGGACATTAAC
AGTAATATAGGGTGGTTGCAGCAGAGACCAGGGAAATCATTTAAG
GGCCTGATCTATCATGGAACCAACTTGGACGATGAAGTTCCATCA
AGGTTCAGTGGCAGTGGATCTGGAGCCGATTATTCTCTCACCATC
AGCAGCCTGGAATCTGAAGATTTTGCAGACTATTACTGTGTACAG
TATGCTCAGTTTCCGTGGACGTTCGGTGGAGGCACCAAGCTGGAA
ATCAAA
anti-EGFRvIII mAb 806 VL aa
SEQ ID: 52
DILMTQSPSSMSVSLGDTVSITCHSSQDINSNIGWLQQRPGKSFK
GLIYHGTNLDDEVPSRFSGSGSGADYSLTISSLESEDFADYYCVQ
YAQFPWTFGGGTKLEIK
anti-CD19 21D4 VH nt
SEQ ID: 53
GAGGTGCAGCTGGTGCAGTCTGGAGCAGAGGTGAAGAAACCAGGA
GAGTCTCTGAAGATCTCCTGTAAGGGTTCTGGATACAGCTTTAGC
AGTTCATGGATCGGCTGGGTGCGCCAGGCACCTGGGAAAGGCCTG
GAATGGATGGGGATCATCTATCCTGATGACTCTGATACCAGATAC
AGTCCATCCTTCCAAGGCCAGGTCACCATCTCAGCCGACAAGTCC
ATCAGGACTGCCTACCTGCAGTGGAGTAGCCTGAAGGCCTCGGAC
ACCGCTATGTATTACTGTGCGAGACATGTTACTATGATTTGGGGA
GTTATTATTGACTTCTGGGGCCAGGGAACCCTGGTCACCGTCTCC
TCA
anti-CD19 21D4 VH aa
SEQ ID: 54
EVQLVQSGAEVKKPGESLKISCKGSGYSFSSSWIGWVRQAPGKGL
EWMGIIYPDDSDTRYSPSFQGQVTISADKSIRTAYLQWSSLKASD
TAMYYCARHVTMIWGVIIDFWGQGTLVTVSS
anti-CD19 21D4 VL nt
SEQ ID: 55
GCCATCCAGTTGACCCAGTCTCCATCCTCCCTGTCTGCATCTGTA
GGAGACAGAGTCACCATCACTTGCCGGGCAAGTCAGGGCATTAGC
AGTGCTTTAGCCTGGTATCAGCAGAAACCAGGGAAAGCTCCTAAG
CTCCTGATCTATGATGCCTCCAGTTTGGAAAGTGGGGTCCCATCA
AGGTTCAGCGGCAGTGGATCTGGGACAGATTTCACTCTCACCATC
AGCAGCCTGCAGCCTGAAGATTTTGCAACTTATTACTGTCAACAG
TTTAATAGTTACCCATTCACTTTCGGCCCTGGGACCAAAGTGGAT
ATCAAA
anti-CD19 21D4 VL aa
SEQ ID: 56
AIQLTQSPSSLSASVGDRVTITCRASQGISSALAWYQQKPGKAPK
LLIYDASSLESGVPSRFSGSGSGTDFTLTISSLQPEDFATYYCQQ
FNSYPFTFGPGTKVDIK
anti-FITC 4-4-20 VH nt
SEQ ID: 57
GAGGTGAAGCTGGATGAGACTGGAGGAGGCTTGGTGCAACCTGGG
AGGCCCATGAAACTCTCCTGTGTTGCCTCTGGATTCACTTTTAGT
GACTACTGGATGAACTGGGTCCGCCAGTCTCCAGAGAAAGGACTG
GAGTGGGTAGCACAAATTAGAAACAAACCTTATAATTATGAAACA
TATTATTCAGATTCTGTGAAAGGCAGATTCACCATCTCAAGAGAT
GATTCCAAAAGTAGTGTCTACCTGCAAATGAACAACTTAAGAGTT
GAAGACATGGGTATCTATTACTGTACGGGTTCTTACTATGGTATG
GACTACTGGGGTCAAGGAACCTCAGTCACCGTCTCCTCA
anti-FITC 4-4-20 VH aa
SEQ ID: 58
EVKLDETGGGLVQPGRPMKLSCVASGFTFSDYWMNWVRQSPEKGL
EWVAQIRNKPYNYETYYSDSVKGRFTISRDDSKSSVYLQMNNLRV
EDMGIYYCTGSYYGMDYWGQGTSVTVSS
anti-FITC 4-4-20 VL nt
SEQ ID: 59
GATGTCGTGATGACCCAAACTCCACTCTCCCTGCCTGTCAGTCTT
GGAGATCAAGCCTCCATCTCTTGCAGATCTAGTCAGAGCCTTGTA
CACAGTAATGGAAACACCTATTTACGTTGGTACCTGCAGAAGCCA
GGCCAGTCTCCAAAGGTCCTGATCTACAAAGTTTCCAACCGATTT
TCTGGGGTCCCAGACAGGTTCAGTGGCAGTGGATCAGGGACAGAT
TTCACACTCAAGATCAGCAGAGTGGAGGCTGAGGATCTGGGAGTT
TATTTCTGCTCTCAAAGTACACATGTTCCGTGGACGTTCGGTGGA
GGCACCAAGCTGGAAATCAAA
anti-FITC 4-4-20 VL aa
SEQ ID: 60
DVVMTQTPLSLPVSLGDQASISCRSSQSLVHSNGNTYLRWYLQKP
GQSPKVLIYKVSNRFSGVPDRFSGSGSGTDFTLKISRVEAEDLGV
YFCSQSTHVPWTFGGGTKLEIK
human IgG1 null (G1m-fa with
ADCC/CDC null mutations) nt
SEQ ID: 61
GCTAGCACCAAGGGCCCATCGGTCTTCCCCCTGGCACCCTCCTCC
AAGAGCACCTCTGGGGGCACAGCGGCCCTGGGCTGCCTGGTCAAG
GACTACTTCCCCGAACCGGTGACGGTGTCGTGGAACTCAGGCGCC
CTGACCAGCGGCGTGCACACCTTCCCGGCTGTCCTACAGTCCTCA
GGACTCTACTCCCTCAGCAGCGTGGTGACCGTGCCCTCCAGCAGC
TTGGGCACCCAGACCTACATCTGCAACGTGAATCACAAGCCCAGC
AACACCAAGGTGGACAAGAGAGTTGAGCCCAAATCTTGTGACAAA
ACTCACACATGCCCACCGTGCCCAGCACCTGAAGCCGCGGGGGCA
CCGTCAGTCTTCCTCTTCCCCCCAAAACCCAAGGACACCCTCATG
ATCTCCCGGACCCCTGAGGTCACATGCGTGGTGGTGGACGTGAGC
CACGAAGACCCTGAGGTCAAGTTCAACTGGTACGTGGACGGCGTG
GAGGTGCATAATGCCAAGACAAAGCCGCGGGAGGAGCAGTACAAC
AGCACGTACCGTGTGGTCAGCGTCCTCACCGTCCTGCACCAGGAC
TGGCTGAATGGCAAGGAGTACAAGTGCGCGGTCTCCAACAAAGCC
CTCCCAGCCCCCATCGAGAAAACCATCTCCAAAGCCAAAGGGCAG
CCCCGAGAACCACAGGTGTACACCCTGCCCCCATCCCGGGATGAG
CTGACCAAGAACCAGGTCAGCCTGACCTGCCTGGTCAAAGGCTTC
TATCCCAGCGACATCGCCGTGGAGTGGGAGAGCAATGGGCAGCCG
GAGAACAACTACAAGACCACGCCTCCCGTGCTGGACTCCGACGGC
TCCTTCTTCCTCTATAGCAAGCTCACCGTGGACAAGAGCAGGTGG
CAGCAGGGGAACGTCTTCTCATGCTCCGTGATGCATGAGGCTCTG
CACAACCACTACACGCAGAAGAGCCTCTCCCTGTCTCCGGGT
human IgG1 null (G1m-fa with
ADCC/CDC null mutations) aa
SEQ ID: 62
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKRVEPKSCDKTHTCPPCPAPEAAGAPSVFLFPPKPKDTLM
ISRTPEVTCVVVDVSHEDPEVKFNWYVDGVEVHNAKTKPREEQYN
STYRVVSVLTVLHQDWLNGKEYKCAVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPG
human Ig Kappa nt
SEQ ID: 63
CGTACGGTGGCTGCACCATCTGTCTTCATCTTCCCGCCATCTGAT
GAGCAGTTGAAATCTGGAACTGCCTCTGTTGTGTGCCTGCTGAAT
AACTTCTATCCCAGAGAGGCCAAAGTACAGTGGAAGGTGGATAAC
GCCCTCCAATCGGGTAACTCCCAGGAGAGTGTCACAGAGCAGGAC
AGCAAGGACAGCACCTACAGCCTCAGCAGCACCCTGACGCTGAGC
AAAGCAGACTACGAGAAACACAAAGTCTACGCCTGCGAAGTCACC
CATCAGGGCCTGAGCTCGCCCGTCACAAAGAGCTTCAACAGGGGA
GAGTGT
human Ig Kappa aa
SEQ ID: 64
RTVAAPSVFIFPPSDEQLKSGTASVVCLLNNFYPREAKVQWKVDN
ALQSGNSQESVTEQDSKDSTYSLSSTLTLSKADYEKHKVYACEVT
HQGLSSPVTKSFNRGEC
SI-35E18 (60C3-L1H1-scFv x PL230C6-
Fab x 323H7-H4L1-scFv x 284A10-
H1L1-scFv) heavy
chain nt
SEQ ID: 65
GACATCCAGATGACCCAGTCTCCTTCCACCCTGTCTGCATCTGTA
GGAGACAGAGTCACCATCACTTGCCAGTCCAGTCAGAGTGTTTAT
AGTAACTGGTTCTCCTGGTATCAGCAGAAACCAGGGAAAGCCCCT
AAGCTCCTGATCTATTCTGCATCCACTCTGGCATCTGGGGTCCCA
TCAAGGTTCAGCGGCAGTGGATCTGGGACAGAATTCACTCTCACC
ATCAGCAGCCTGCAGCCTGATGATTTTGCAACTTATTACTGCGCA
GGCGGTTACAATACTGTTATTGATACTTTTGCTTTCGGCGGAGGG
ACCAAGGTGGAGATCAAAGGCGGTGGCGGTAGTGGGGGAGGCGGT
TCTGGCGGCGGAGGGTCCGGCGGTGGAGGATCAGAGGTGCAGCTG
TTGGAGTCTGGGGGAGGCTTGGTACAGCCTGGGGGGTCCCTGAGA
CTCTCCTGTGCAGCCTCTGGAATCGACTTCAGTAGGAGATACTAC
ATGTGCTGGGTCCGCCAGGCTCCAGGGAAGGGGCTGGAGTGGATC
GCATGCATATATACTGGTAGCCGCGATACTCCTCACTACGCGAGC
TCCGCGAAAGGCCGGTTCACCATCTCCAGAGACAATTCCAAGAAC
ACGCTGTATCTGCAAATGAACAGCCTGAGAGCCGAGGACACGGCC
GTATATTACTGTGCGAGAGAAGGTAGCCTGTGGGGCCAGGGAACC
CTGGTCACCGTCTCGAGCGGCGGTGGAGGGTCCGGCGGTGGTGGA
TCCCAGTCGGTGGAGGAGTCTGGGGGAGGCTTGGTCCAGCCTGGG
GGGTCCCTGAGACTCTCCTGTACAGCCTCTGGAATCGACCTTAAT
ACCTACGACATGATCTGGGTCCGCCAGGCTCCAGGCAAGGGGCTA
GAGTGGGTTGGAATCATTACTTATAGTGGTAGTAGATACTACGCG
AACTGGGCGAAAGGCCGATTCACCATCTCCAAAGACAATACCAAG
AACACGGTGTATCTGCAAATGAACAGCCTGAGAGCTGAGGACACG
GCTGTGTATTACTGTGCCAGAGATTATATGAGTGGTTCCCACTTG
TGGGGCCAGGGAACCCTGGTCACCGTCTCTAGTGCTAGCACCAAG
GGCCCATCGGTCTTCCCCCTGGCACCCTCCTCCAAGAGCACCTCT
GGGGGCACAGCGGCCCTGGGCTGCCTGGTCAAGGACTACTTCCCC
GAACCGGTGACGGTGTCGTGGAACTCAGGCGCCCTGACCAGCGGC
GTGCACACCTTCCCGGCTGTCCTACAGTCCTCAGGACTCTACTCC
CTCAGCAGCGTGGTGACCGTGCCCTCCAGCAGCTTGGGCACCCAG
ACCTACATCTGCAACGTGAATCACAAGCCCAGCAACACCAAGGTG
GACAAGAGAGTTGAGCCCAAATCTTGTGACAAAACTCACACATGC
CCACCGTGCCCAGCACCTGAAGCCGCGGGGGCACCGTCAGTCTTC
CTCTTCCCCCCAAAACCCAAGGACACCCTCATGATCTCCCGGACC
CCTGAGGTCACATGCGTGGTGGTGGACGTGAGCCACGAAGACCCT
GAGGTCAAGTTCAACTGGTACGTGGACGGCGTGGAGGTGCATAAT
GCCAAGACAAAGCCGCGGGAGGAGCAGTACAACAGCACGTACCGT
GTGGTCAGCGTCCTCACCGTCCTGCACCAGGACTGGCTGAATGGC
AAGGAGTACAAGTGCGCGGTCTCCAACAAAGCCCTCCCAGCCCCC
ATCGAGAAAACCATCTCCAAAGCCAAAGGGCAGCCCCGAGAACCA
CAGGTGTATACCCTGCCCCCATCCCGGGATGAGCTGACCAAGAAC
CAGGTCAGCCTGACCTGCCTGGTCAAAGGCTTCTATCCCAGCGAC
ATCGCCGTGGAGTGGGAGAGCAATGGGCAGCCGGAGAACAACTAC
AAGACCACGCCTCCCGTGCTGGACTCCGACGGCTCCTTCTTCCTC
TATAGCAAGCTCACCGTGGACAAGAGCAGGTGGCAGCAGGGGAAC
GTCTTCTCATGCTCCGTGATGCATGAGGCTCTGCACAACCACTAC
ACGCAGAAGAGCCTCTCCCTGTCTCCGGGTGGCGGTGGAGGGTCC
GGCGGTGGTGGATCCGAGGTGCAGCTGTTGGAGTCTGGGGGAGGC
TTGGTACAGCCTGGGGGGTCCCTGAGACTCTCCTGTGCAGCCTCT
GGATTCACCATCAGTCGCTACCACATGACTTGGGTCCGCCAGGCT
CCAGGGAAGGGGCTGGAGTGGATCGGACATATTTATGTTAATAAT
GATGACACAGACTACGCGAGCTCCGCGAAAGGCCGGTTCACCATC
TCCAGAGACAATTCCAAGAACACGCTGTATCTGCAAATGAACAGC
CTGAGAGCCGAGGACACGGCCACCTATTTCTGTGCGAGATTGGAT
GTTGGTGGTGGTGGTGCTTATATTGGGGACATCTGGGGCCAGGGA
ACTCTGGTTACCGTCTCTTCAGGCGGTGGCGGTAGTGGGGGAGGC
GGTTCTGGCGGCGGAGGGTCCGGCGGTGGAGGATCAGACATCCAG
ATGACCCAGTCTCCATCCTCCCTGTCTGCATCTGTAGGAGACAGA
GTCACCATCACTTGCCAGTCCAGTCAGAGTGTTTATAACAACAAC
GACTTAGCCTGGTATCAGCAGAAACCAGGGAAAGTTCCTAAGCTC
CTGATCTATTATGCTTCCACTCTGGCATCTGGGGTCCCATCTCGG
TTCAGTGGCAGTGGATCTGGGACAGATTTCACTCTCACCATCAGC
AGCCTGCAGCCTGAAGATGTTGCAACTTATTACTGTGCAGGCGGT
TATGATACGGATGGTCTTGATACGTTTGCTTTCGGCGGAGGGACC
AAGGTGGAGATCAAAGGCGGTGGAGGGTCCGGCGGTGGTGGATCC
GAGGTGCAGCTGGTGGAGTCTGGGGGAGGCTTGGTCCAGCCTGGG
GGGTCCCTGAGACTCTCCTGTGCAGCCTCTGGATTCACCATCAGT
ACCAATGCAATGAGCTGGGTCCGCCAGGCTCCAGGGAAGGGGCTG
GAGTGGATCGGAGTCATTACTGGTCGTGATATCACATACTACGCG
AGCTGGGCGAAAGGCAGATTCACCATCTCCAGAGACAATTCCAAG
AACACGCTGTATCTTCAAATGAACAGCCTGAGAGCCGAGGACACG
GCTGTGTATTACTGTGCGCGCGACGGTGGATCATCTGCTATTACT
AGTAACAACATTTGGGGCCAAGGAACTCTGGTCACCGTTTCTTCA
GGCGGTGGCGGTAGTGGGGGAGGCGGTTCTGGCGGCGGAGGGTCC
GGCGGTGGAGGATCAGACGTCGTGATGACCCAGTCTCCTTCCACC
CTGTCTGCATCTGTAGGAGACAGAGTCACCATCAATTGCCAAGCC
AGTGAGAGCATTAGCAGTTGGTTAGCCTGGTATCAGCAGAAACCA
GGGAAAGCCCCTAAGCTCCTGATCTATGAAGCATCCAAACTGGCA
TCTGGGGTCCCATCAAGGTTCAGCGGCAGTGGATCTGGGACAGAG
TTCACTCTCACCATCAGCAGCCTGCAGCCTGATGATTTTGCAACT
TATTACTGCCAAGGCTATTTTTATTTTATTAGTCGTACTTATGTA
AATTCTTTCGGCGGAGGGACCAAGGTGGAGATCAAA
SI-35E18 (460C3-L1H1-scFv x PL230C6-
Fab x 323H7-H4L1-scFv x 284A10-
H1L1-scFv) heavy chain aa
SEQ ID: 66
DIQMTQSPSTLSASVGDRVTITCQSSQSVYSNWFSWYQQKPGKAP
KLLIYSASTLASGVPSRFSGSGSGTEFTLTISSLQP
DDFATYYCAGGYNTVIDTFAFGGGTKVEIK
(anti-4-1BB 460C3 VLv1)
GGGGSGGGGSGGGGSGGGGS
(Gly₄Ser) × 4 linker
EVQLLESGGGLVQPGGSLRLSCAASGIDFSRRYYMCWVRQAPGKG
LEWIACIYTGSRDTPHYASSAKGRFTISRDNSKNTLYLQMNSLRA
EDTAVYYCAREGSLWGQGTLVTVSS
(anti-4-1BB 460C3 VHv1)
GGGGSGGGGS
(Gly₄Ser) × 2 linker
QSVEESGGGLVQPGGSLRLSCTASGIDLNTYDMIWVRQAPGKGLE
WVGIITYSGSRYYANWAKGRFTISKDNTKNTVYLQMNSLRAEDTA
VYYCARDYMSGSHLWGQGTLVTVSS
(anti-PD-L1 PL230C6 VHv3)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKRVEPKSCDKTHTCPPCPAPEAAGAPSVFLFPPKPKDTLM
ISRTPEVTCVVVDVSHEDPEVKFNWYVDGVEVHNAKTKPREEQYN
STYRVVSVLTVLHQDWLNGKEYKCAVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPG
(human IgG1 null)
GGGGSGGGGS
(Gly₄Ser) × 2 linker
EVQLLESGGGLVQPGGSLRLSCAASGFTISRYHMTWVRQAPGKGL
EWIGHIYVNNDDTDYASSAKGRFTISRDNSKNTLYLQMNSLRAED
TATYFCARLDVGGGGAYIGDIWGQGTLVTVSS
(anti-ROR1 323H7 VHv4)
GGGGSGGGGSGGGGSGGGGS
(Gly₄Ser) × 4 linker
DIQMTQSPSSLSASVGDRVTITCQSSQSVYNNNDLAWYQQKPGKV
PKLLIYYASTLASGVPSRFSGSGSGTDFTLTISSLQPEDVATYYC
AGGYDTDGLDTFAFGGGTKVEIK
(anti-ROR1 323H7 VLv1)
GGGGSGGGGS
(Gly₄Ser) × 2 linker
EVQLVESGGGLVQPGGSLRLSCAASGFTISTNAMSWVRQAPGKGL
EWIGVITGRDITYYASWAKGRFTISRDNSKNTLYLQMNSLRAEDT
AVYYCARDGGSSAITSNNIWGQGTLVTVSS
(anti-CD3 284A10 VHv1)
GGGGSGGGGSGGGGSGGGGS
(Gly₄Ser) × 4 linker
DVVMTQSPSTLSASVGDRVTINCQASESISSWLAWYQQKPGKAPK
LLIYEASKLASGVPSRFSGSGSGTEFTLTISSLQPDDFATYYCQG
YFYFISRTYVNSFGGGTKVEIK
(anti-CD3 284A10 VLv1)
SI-35E18 (460C3-L1H1-scFv x PL230C6-
Fab x 323H7-H4L1-scFv x 284A10-
H1L1-scFv) light chain nt
SEQ ID: 67
GCCTATGATATGACCCAGTCTCCATCTTCCGTGTCTGCATCTGTA
GGAGACAGAGTCACCATCAAGTGTCAGGCCAGTGAGGACATTTAT
AGCTTCTTGGCCTGGTATCAGCAGAAACCAGGGAAAGCCCCTAAG
CTCCTGATCCATTCTGCATCCTCTCTGGCATCTGGGGTCCCATCA
AGGTTCAGCGGCAGTGGATCTGGGACAGATTTCACTCTCACCATC
AGCAGCCTGCAGCCTGAAGATTTTGCAACTTACTATTGTCAACAG
GGTTATGGTAAAAATAATGTTGATAATGCTTTCGGCGGAGGGACC
AAGGTGGAGATCAAACGTACGGTGGCTGCACCATCTGTCTTCATC
TTCCCGCCATCTGATGAGCAGTTGAAATCTGGAACTGCCTCTGTT
GTGTGCCTGCTGAATAACTTCTATCCCAGAGAGGCCAAAGTACAG
TGGAAGGTGGATAACGCCCTCCAATCGGGTAACTCCCAGGAGAGT
GTCACAGAGCAGGACAGCAAGGACAGCACCTACAGCCTCAGCAGC
ACCCTGACGCTGAGCAAAGCAGACTACGAGAAACACAAAGTCTAC
GCCTGCGAAGTCACCCATCAGGGCCTGAGCTCGCCCGTCACAAAG
AGCTTCAACAGGGGAGAGTGT
SI-35E18 (460C3-L1H1-scFv x PL230C6-
Fab x 323H7-H4L1-scFv x 284A10-
H1L1-scFv) light chain aa
SEQ ID: 68
AYDMTQSPSSVSASVGDRVTIKCQASEDIYSFLAWYQQKPGKAPK
LLIHSASSLASGVPSRFSGSGSGTDFTLTISSLQPEDFATYYCQQ
GYGKNNVDNAFGGGTKVEIK
(anti-PD-L1 PL230C6 VLv2)
RTVAAPSVFIFPPSDEQLKSGTASVVCLLNNFYPREAKVQWKVDN
ALQSGNSQESVTEQDSKDSTYSLSSTLTLSKADYEKHKVYACEVT
HQGLSSPVTKSFNRGEC
(human Ig Kappa)

### Other Sequences:

| antigen | antibody | VH/VL | organism | patent | seq ID | Company |
|---|---|---|---|---|---|---|
| FITC | 4-4-20 | VH | artificial | US 2009/0042291 A1 | 14 | Xencor |
| FITC | 4-4-20 | VL | artificial | US 2009/0042291 A1 | 13 | Xencor |
| CD19 | 21D4 | VH | human | US 2009/0142349 A1 | 1 | Medarex |
| CD19 | 21D4 | VL | human | US 2009/0142349 A1 | 9 | Medarex |
| EGFRvIII | mAb 806 | VH | mouse | US 2009/0137782 A1 | 11 | Ludwig Institute |
| EGFRvIII | mAb 806 | VL | mouse | US 2009/0137782 A1 | 12 | Ludwig Institute |

NOTE:
EGFRvIII Systimmune sequence missing C-terminal Arginine.

### Sequence Index:

| SEQ ID | Description |
|---|---|
| 1 | anti-CD3 284A10 VHv1 nt |
| 2 | anti-CD3 284A10 VHv1 aa |
| 3 | anti-CD3 284A10 VLv1 nt |
| 4 | anti-CD3 284A10 VLv1 aa |
| 5 | anti-CD3 480C8 VHv1 nt |
| 6 | anti-CD3 480C8 VHv1 aa |
| 7 | anti-CD3 480C8 VLv1 nt |
| 8 | anti-CD3 480C8 VLv1 aa |
| 9 | anti-PD-L1 PL230C6 VHv3 nt |
| 10 | anti-PD-L1 PL230C6 VHv3 aa |
| 11 | anti-PD-L1 PL230C6 VLv2 nt |
| 12 | anti-PD-L1 PL230C6 VLv2 aa |
| 13 | anti-PD-L1 PL221G5 VHv1 nt |
| 14 | anti-PD-L1 PL221G5 VHv1 aa |
| 15 | anti-PD-L1 PL221G5 VLv1 nt |
| 16 | anti-PD-L1 PL221G5 VLv1 aa |
| 17 | anti-PD-1 PD224D1 VHv2 nt |
| 18 | anti-PD-1 PD224D1 VHv2 aa |
| 19 | anti-PD-1 PD224D1 VLv2 nt |
| 20 | anti-PD-1 PD224D1 VLv2 aa |
| 21 | anti-4-IBB 420H5 VHv3 nt |
| 22 | anti-4-IBB 420H5 VHv3 aa |
| 23 | anti-4-IBB 420H5 VLv3 nt |
| 24 | anti-4-IBB 420H5 VHLv3 aa |
| 25 | anti-4-IBB 466F6 VHv2 nt |
| 26 | anti-4-IBB 466F6 VHv2 aa |
| 27 | anti-4-IBB 466F6 VLv5 nt |
| 28 | anti-4-IBB 466F6 VLv5 aa |
| 29 | anti-4-IBB 460C3 VHv1 nt |
| 30 | anti-4-IBB 460C3 VHv1 aa |
| 31 | anti-4-IBB 460C3 VLv1 nt |
| 32 | anti-4-IBB 460C3 VLv1 aa |
| 33 | anti-ROR1 324C6 VHv2 nt |
| 34 | anti-ROR1 324C6 VHv2 aa |
| 35 | anti-ROR1 324C6 VLv1 nt |
| 36 | anti-ROR1 324C6 VLv1 aa |
| 37 | anti-ROR1 323H7 VHv4 nt |
| 38 | anti-ROR1 323H7 VHv4 aa |
| 39 | anti-ROR1 323H7 VLv1 nt |
| 40 | anti-ROR1 323H7 VLv1 aa |
| 41 | anti-ROR1 338H4 VHv3 nt |
| 42 | anti-ROR1 338H4 VHv3 aa |
| 43 | anti-ROR1 338H4 VLv4 nt |
| 44 | anti-ROR1 338H4 VLv4 aa |
| 45 | anti-ROR1 330F11 VHv1 nt |
| 46 | anti-ROR1 330F11 VHv1 aa |
| 47 | anti-ROR1 330F11 VLv1 nt |
| 48 | anti-ROR1 330F11 VLv1 aa |
| 49 | anti-EGFRvIII mAb 806 VH nt |
| 50 | anti-EGFRvIII mAb 806 VH aa |
| 51 | anti-EGFRvIII mAb 806 VL nt |
| 52 | anti-EGFRvIII mAb 806 VL aa |
| 53 | anti-CD19 21D4 VH nt |
| 54 | anti-CD19 21D4 VH aa |
| 55 | anti-CD19 21D4 VL nt |
| 56 | anti-CD19 21D4 VL aa |
| 57 | anti-FITC 4-4-20 VH nt |
| 58 | anti-FITC 4-4-20 VH aa |
| 59 | anti-FITC 4-4-20 VL nt |
| 60 | anti-FITC 4-4-20 VL aa |
| 61 | human IgG1 null (G1m-fa with ADCC/CDC null mutations) nt |
| 62 | human IgG1 null (G1m-fa with ADCC/CDC null mutations) aa |
| 63 | human Ig Kappa nt |
| 64 | human Ig Kappa aa |
| 65 | SI-35E18 (60C3-L1H1-scFv × PL230C6-Fab × |
| | 323H7-H4L1-scFv × 284A10-H1L1-scFv) heavy chain nt |
| 66 | SI-35E18 (60C3-L1H1-scFv × PL230C6-Fab × |
| | 323H7-H4L1-scFv × 284A10-H1L1-scFv) heavy chain aa |
| 67 | SI-35E18 (60C3-L1H1-scFv × PL230C6-Fab × |
| | 323H7-H4L1-scFv × 284A10-H1L1-scFv) light chain nt |
| 68 | SI-35E18 (60C3-L1H1-scFv × PL230C6-Fab × |
| | 323H7-H4L1-scFv × 284A10-H1L1-scFv) light chain aa |

1. A tetra-specific antibody monomer having a N-terminal and a C-terminal, comprising in tandem from the N-terminal to the C-terminal,
a first scFv domain at the N-terminal, a Fab domain,
a Fc domain,
a second scFv domain, and
a third scFv at the C-terminal,
wherein the first scFv domain, the Fab domain, the second scFv domain, and the third scFv domain each has a binding specificity against a different antigen, and wherein the antigen is a tumor antigen, an immune signaling antigen, or a combination thereof, and
wherein one of the first scFv domain, the Fab domain, the second scFv domain, and the third scFv domain has a binding specificity against CD3 and comprises 3 CDRs of an amino acid sequence set forth in SEQ ID NO: 2 and 3 CDRs of an amino acid sequence set forth in SEQ ID NO: 4.

2. The tetra-specific antibody monomer of claim 1, wherein the first scFv domain, the Fab domain, the second scFv domain, and the third scFv domain each independently has a binding specificity against an antigen selected from ROR1, PD-L1, CD3, 4-1BB, EGFRvIII, and CD19, and wherein the Fc domain comprises a human IgG Fc domain.

3. (canceled)

4. The tetra-specific antibody monomer of claim 1, wherein the first scFv domain has a binding specificity against CD3, and wherein the Fab domain has a binding specificity against EGRF VIII, and comprises 3 CDRs of an amino acid sequence set forth in SEQ ID NO: 50 and 3 CDRs of an amino acid sequence set forth in SEQ ID NO: 52; the second scFv domain has a binding specificity against PD-L1, and comprises 3 CDRs of an amino acid sequence set forth in SEQ ID NO: 14 and 3 CDRs of an amino acid sequence set forth in SEQ ID NO: 16; and the third scFv domain has a binding specificity against 4-1BB, and comprises 3 CDRs of an amino acid sequence set forth in SEQ ID NO: 22 and 3 CDRs of an amino acid sequence set forth in SEQ ID NO: 24.

5. The tetra-specific antibody monomer of claim 1, wherein the Fab domain has a binding specificity against CD3, and wherein the first scFv domain has a binding specificity against CD19, and comprises 3 CDRs of an amino acid sequence set forth in SEQ ID NO: 54 and 3 CDRs of an amino acid sequence set forth in SEQ ID NO: 56; the second scFv domain has a binding specificity against 4-1BB, and comprises 3 CDRs of an amino acid sequence set forth in SEQ ID NO: 26 and 3 CDRs of an amino acid sequence set forth in SEQ ID NO: 28; and the third scFv domain has a binding specificity against PD-L1, and comprises 3 CDRs of an amino acid sequence set forth in SEQ ID NO: 14 and 3 CDRs of an amino acid sequence set forth in SEQ ID NO: 16.

6. The tetra-specific antibody monomer of claim 1, wherein the second scFv domain has a binding specificity against CD3, and wherein
the first scFv domain has a binding specificity against 4-1BB, and comprises 3 CDRs of an amino acid sequence set forth in SEQ ID NO: 26 and 3 CDRs of an amino acid sequence set forth in SEQ ID NO: 28; the Fab domain has a binding specificity against PD-L1, and comprises 3 CDRs of an amino acid sequence set forth in SEQ ID NO: 10 and 3 CDRs of an amino acid sequence set forth in SEQ ID NO: 12; and the third scFv domain has a binding specificity against CD19, and comprises 3 CDRs of an amino acid sequence set forth in SEQ ID NO: 54 and 3 CDRs of an amino acid sequence set forth in SEQ ID NO: 56; or
the first scFv domain has a binding specificity against 4-1BB, and comprises 3 CDRs of an amino acid sequence set forth in SEQ ID NO: 26 and 3 CDRs of an amino acid sequence set forth in SEQ ID NO: 28; the Fab domain has a binding specificity against CD19, and comprises 3 CDRs of an amino acid sequence set forth in SEQ ID NO: 54 and 3 CDRs of an amino acid sequence set forth in SEQ ID NO: 56; and the third scFv domain has a binding specificity against PD-L1, and comprises 3 CDRs of an amino acid sequence set forth in SEQ ID NO: 14 and 3 CDRs of an amino acid sequence set forth in SEQ ID NO: 16.

7. The tetra-specific antibody monomer of claim 1, wherein the third scFv domain has a binding specificity against CD3, and wherein,
the first scFv domain has a binding specificity against 4-1BB, and comprises 3 CDRs of an amino acid sequence set forth in SEQ ID NO: 26 and 3 CDRs of an amino acid sequence set forth in SEQ ID NO: 28; the Fab domain has a binding specificity against PD-L1, and comprises 3 CDRs of an amino acid sequence set forth in SEQ ID NO: 10 and 3 CDRs of an amino acid sequence set forth in SEQ ID NO: 12; the second scFv domain has a binding specificity against ROR1, and comprises 3 CDRs of an amino acid sequence set forth in SEQ ID NO: 38 and 3 CDRs of an amino acid sequence set forth in SEQ ID NO: 40 (SI-35E20);
the first scFv domain has a binding specificity against 4-1BB, and comprises 3 CDRs of an amino acid sequence set forth in SEQ ID NO: 30 and 3 CDRs of an amino acid sequence set forth in SEQ ID NO: 32; the Fab domain has a binding specificity against PD-L1, and comprises 3 CDRs of an amino acid sequence set forth in SEQ ID NO: 10 and 3 CDRs of an amino acid sequence set forth in SEQ ID NO: 12; the second scFv domain has a binding specificity against ROR1, and comprises 3 CDRs of an amino acid sequence set forth in SEQ ID NO: 38 and 3 CDRs of an amino acid sequence set forth in SEQ ID NO: 40 (SI-35E18);
the first scFv domain has a binding specificity against PD-L1, and comprises 3 CDRs of an amino acid sequence set forth in SEQ ID NO: 10 and 3 CDRs of an amino acid sequence set forth in SEQ ID NO: 12; the Fab domain has a binding specificity against 4-1BB, and comprises 3 CDRs of an amino acid sequence set forth in SEQ ID NO: 26 and 3 CDRs of an amino acid sequence set forth in SEQ ID NO: 28; the second scFv domain has a binding specificity against CD19, and comprises 3 CDRs of an amino acid sequence set forth in SEQ ID NO: 54 and 3 CDRs of an amino acid sequence set forth in SEQ ID NO: 56;
the first scFv domain has a binding specificity against 4-1BB, and comprises 3 CDRs of an amino acid sequence set forth in SEQ ID NO: 30 and 3 CDRs of an amino acid sequence set forth in SEQ ID NO: 32; the Fab domain has a binding specificity against PD-L1, and comprises 3 CDRs of an amino acid sequence set forth in SEQ ID NO: 10 and 3 CDRs of an amino acid sequence set forth in SEQ ID NO: 12; the second scFv domain has a binding specificity against ROR1, and comprises 3 CDRs of an amino acid sequence set forth in SEQ ID NO: 38 and 3 CDRs of an amino acid sequence set forth in SEQ ID NO: 40;
the first scFv domain has a binding specificity against 4-1BB, and comprises 3 CDRs of an amino acid sequence set forth in SEQ ID NO: 26 and 3 CDRs of an amino acid sequence set forth in SEQ ID NO: 28; the Fab domain has a binding specificity against PD-L1, and comprises 3 CDRs of an amino acid sequence set forth in SEQ ID NO: 10 and 3 CDRs of an amino acid sequence set forth in SEQ ID NO: 12; the second scFv domain has a binding specificity against ROR1, and comprises 3 CDRs of an amino acid sequence set forth in SEQ ID NO: 38 and 3 CDRs of an amino acid sequence set forth in SEQ ID NO: 40; or
the first scFv domain has a binding specificity against PD-L1, and comprises 3 CDRs of an amino acid sequence set forth in SEQ ID NO: 18 and 3 CDRs of an amino acid sequence set forth in SEQ ID NO: 20; the Fab domain has a binding specificity against 4-1BB, and comprises 3 CDRs of an amino acid sequence set forth in SEQ ID NO: 26 and 3 CDRs of an amino acid sequence set forth in SEQ ID NO: 28; the second scFv domain has a binding specificity against CD19, and comprises 3 CDRs of an amino acid sequence set forth in SEQ ID NO: 54 and 3 CDRs of an amino acid sequence set forth in SEQ ID NO: 56.

8. (canceled)

9. The tetra-specific antibody monomer of claim 1, wherein the first scFv domain, the second scFv domain, or the third scFv domain comprises a gly-gly-gly-gly-ser (G4S)n linker, wherein n is 2, 3 or 4.

10. The tetra-specific antibody monomer of claim 1, comprising an amino acid sequence having at least 98% sequence identity to SEQ ID NO. 02, 04, 06, 08, 10, 12, 14, 16, 18, 20, 22, 24, 26, 28, 30, 32, 34, 36, 38, 40, 42, 44, 46, 48, 50, 52, 54, 56, 58, 60, 62, or 64.

11. A binding domain for the tetra-specific antibody monomer of claim 1, comprising an amino acid sequence having at least 98% sequence identity to SEQ ID NO. 02, 04, 06, 08, 10, 12, 14, 16, 18, 20, 22, 24, 26, 28, 30, 32, 34, 36, 38, 40, 42, 44, 46, 48, 50, 52, 54, 56, 58, or 60.

12. (canceled)

13. A tetra-specific antibody, comprising a tetra-specific antibody monomer of claim 1.

14. The tetra-specific antibody of claim 13, comprising an amino acid sequence having at least 98% sequence identity to SEQ ID NO. 66, and 68.

15. An isolated nucleic acid sequence, encoding an amino acid sequence having at least 98% sequence identity to SEQ ID NO. 02, 04, 06, 08, 10, 12, 14, 16, 18, 20, 22, 24, 26, 28, 30, 32, 34, 36, 38, 40, 42, 44, 46, 48, 50, 52, 66 or 68.

16. An expression vector, comprising the isolated nucleic acid sequence of claim 15.

17. A host cell comprising the isolated nucleic acid sequence of claim 15, wherein the host cell is a prokaryotic cell or a eukaryotic cell.

18. A method for producing a tetra-specific antibody of claim 13, comprising culturing a host cell comprising an isolated nucleic acid sequence such that the DNA sequence encoding the tetra-specific antibody or monomer is expressed, and purifying said tetra-specific antibody.

19. A method for treating or preventing a cancer, said method comprising administering a pharmaceutical composition comprising a purified tetra-specific antibody of claim 13.

20. An immuno-conjugate comprising a cytotoxic agent or an imaging agent linked to the tetra-specific antibody of claim 13 through a linker, wherein the linker comprises an ester bond, an ether bond, an amid bond, a disulphide bond, an imide bond, a sulfone bond, a phosphate bond, a phosphorus ester bond, a peptide bond, a hydrophobic poly(ethylene glycol) linker, or a combination thereof.

21. (canceled)

22. A pharmaceutical composition, comprising a pharmaceutically acceptable carrier and the tetra-specific antibody of claim 13.

23. The pharmaceutical composition of claim 22, further comprising a therapeutic agent selected from a radioisotope, radionuclide, a toxin, a chemotherapeutic agent or a combination thereof.

24. A method of treating a human subject with a cancer, comprising administering to the subject an effective amount of the tetra-specific antibody according to claim 13.

25-26. (canceled)
